SEQUENCE LISTING (77 sequences)

SEQ ID NO: 1 - DNA, 23 bp, Artificial Sequence
  VH1B/7A-Back primer
cagrtgcagctggtgcartctgg

SEQ ID NO: 2 - DNA, 23 bp, Artificial Sequence
  VH1C-Back primer
saggtccagctggtrcagtctgg

SEQ ID NO: 3 - DNA, 23 bp, Artificial Sequence
  VH3B-Back primer
saggtgcagctggtggagtctgg

SEQ ID NO: 4 - DNA, 23 bp, Artificial Sequence
  VH5B-Back primer
gargtgcagctggtgcagtctgg

SEQ ID NO: 5 - DNA, 23 bp, Artificial Sequence
  VH4C-Back primer
cagstgcagctgcaggagtcsgg

SEQ ID NO: 6 - DNA, 23 bp, Artificial Sequence
  VH6A-Back primer
caggtacagctgcagcagtcagg

SEQ ID NO: 7 - DNA, 23 bp, Artificial Sequence
  VH2B-Back primer
cagrtcaccttgaaggagtctgg

SEQ ID NO: 8 - DNA, 23 bp, Artificial Sequence
  VH4B-Back primer
caggtgcagctgcagcagtgggg

SEQ ID NO: 9 - DNA, 24 bp, Artificial Sequence
  Hu-IgG1-CH1-For primer
gtccaccttggtgttgctgggctt

SEQ ID NO: 10 - DNA, 24 bp, Artificial Sequence
  Hu-IgM-CH1-For primer
tggaagaggcacgttcttttcttt

SEQ ID NO: 11 - DNA, 56 bp, Artificial Sequence
  VH1B/7A-SfiI-Back primer
gtcctcgcaactgcggcccagccggccatggcccagrtgcagctggtgcartctgg

SEQ ID NO: 12 - DNA, 56 bp, Artificial Sequence
  VH1C-SfiI-Back primer
gtcctcgcaactgcggcccagccggccatggccsaggtccagctggtrcagtctgg

SEQ ID NO: 13 - DNA, 56 bp, Artificial Sequence
  VH3B-SfiI-Back primer
gtcctcgcaactgcggcccagccggccatggccsaggtgcagctggtggagtctgg

SEQ ID NO: 14 - DNA, 56 bp, Artificial Sequence
  VH5B-SfiI-Back primer
gtcctcgcaactgcggcccagccggccatggccgargtgcagctggtgcagtctgg

SEQ ID NO: 15 - DNA, 56 bp, Artificial Sequence
  VH4C-SfiI-Back primer
gtcctcgcaactgcggcccagccggccatggcccagstgcagctgcaggagtcsgg

SEQ ID NO: 16 - DNA, 56 bp, Artificial Sequence
  VH6A-SfiI-Back primer
gtcctcgcaactgcggcccagccggccatggcccaggtacagctgcagcagtcagg

SEQ ID NO: 17 - DNA, 56 bp, Artificial Sequence
  VH2B-SfiI-Back primer
gtcctcgcaactgcggcccagccggccatggcccagrtcaccttgaaggagtctgg

SEQ ID NO: 18 - DNA, 56 bp, Artificial Sequence
  VH4B-SfiI-Back primer
gtcctcgcaactgcggcccagccggccatggcccaggtgcagctgcagcagtgggg

SEQ ID NO: 19 - DNA, 48 bp, Artificial Sequence
  JH1/2-NotI-For primer
gagtcattctcgacttgcggccgctgaggagacggtgaccagggtgcc

SEQ ID NO: 20 - DNA, 48 bp, Artificial Sequence
  JH4/5-NotI-For primer
gagtcattctcgacttgcggccgctgaggagacggtgaccagggttcc

SEQ ID NO: 21 - DNA, 48 bp, Artificial Sequence
  JH3-NotI-For primer
gagtcattctcgacttgcggccgctgaagagacggtgaccattgtccc

SEQ ID NO: 22 - DNA, 48 bp, Artificial Sequence
  JH6-NotI-For primer
gagtcattctcgacttgcggccgctgaggagacggtgaccgtggtccc

SEQ ID NO: 23 - DNA, 22 bp, Artificial Sequence
  Exback1 primer
gacacggccgtntattactgtg

SEQ ID NO: 24 - DNA, 22 bp, Artificial Sequence
  Exback2 primer
gacacggccgtntattattgtg

SEQ ID NO: 25 - DNA, 22 bp, Artificial Sequence
  Exback3 primer
gacacggctgtrtatttctgtg

SEQ ID NO: 26 - DNA, 21 bp, Artificial Sequence
  Exfor1 primer
gaccagggtbccctggcccca

SEQ ID NO: 27 - DNA, 21 bp, Artificial Sequence
  Exfor2 primer
gaccgtggtyccttggcccca

SEQ ID NO: 28 - DNA, 21 bp, Artificial Sequence
  Exfor3 primer
gaccagggtgccacggcccca

SEQ ID NO: 29 - DNA, 55 bp, Artificial Sequence
  Exback1-sfiI primer
gtcctcgcaactgcggcccagccggccatggccgacacggccgtntattactgtg

SEQ ID NO: 30 - DNA, 55 bp, Artificial Sequence
  Exback2-sfiI primer
gtcctcgcaactgcggcccagccggccatggccgacacggccgtntattattgtg

SEQ ID NO: 31 - DNA, 55 bp, Artificial Sequence
  Exback3-sfiI primer
gtcctcgcaactgcggcccagccggccatggccgacacggctgtrtatttctgtg

SEQ ID NO: 32 - DNA, 57 bp, Artificial Sequence
  ExFor1-NotI primer
gagtcattctcgacttgcggccgctgaaccgcctccgaccagggtbccctggcccca

SEQ ID NO: 33 - DNA, 57 bp, Artificial Sequence
  ExFor2-NotI primer
gagtcattctcgacttgcggccgctgaaccgcctccgaccgtggtyccttggcccca

SEQ ID NO: 34 - DNA, 57 bp, Artificial Sequence
  ExFor3-NotI primer
gagtcattctcgacttgcggccgctgaaccgcctccgaccagggtgccacggcccca

SEQ ID NO: 35 - DNA, 23 bp, Artificial Sequence
  Degenerated framework 1 primer ABL013
gaggtbcarctgcaggastcygg

SEQ ID NO: 36 - DNA, 53 bp, Artificial Sequence
  hinge primer
aacagttaagcttccgcttgcggccgcggagctggggtcttcgctgtggtgcg

SEQ ID NO: 37 - DNA, 53 bp, Artificial Sequence
  hinge primer
aacagttaagcttccgcttgcggccgctggttgtggttttggtgtcttgggtt

SEQ ID NO: 38 - DNA, 20 bp, Artificial Sequence
  Exback1 primer
gacacggccgbctattactg

SEQ ID NO: 39 - DNA, 20 bp, Artificial Sequence
  Exback2 primer
gacacggccgtttatwactg

SEQ ID NO: 40 - DNA, 20 bp, Artificial Sequence
  Exback3 primer
gacacggccgtgtattaytg

SEQ ID NO: 41 - DNA, 20 bp, Artificial Sequence
  Exback4 primer
gacacggccgtctattwttg

SEQ ID NO: 42 - DNA, 20 bp, Artificial Sequence
  Exback5 primer
gacacggccgwttattattg

SEQ ID NO: 43 - DNA, 20 bp, Artificial Sequence
  Exback6 primer
gacacggccatytattwctg

SEQ ID NO: 44 - DNA, 20 bp, Artificial Sequence
  Exback7 primer
gacacgggactytattactg

SEQ ID NO: 45 - protein, 7 aa, Artificial Sequence
  amino acid sequence for FR3 backward primers
DTAVYYC

SEQ ID NO: 46 - DNA, 20 bp, Artificial Sequence
  FR4 A primer
ggggccagggvacycaggtc

SEQ ID NO: 47 - DNA, 20 bp, Artificial Sequence
  Exfor1 primer
gacctgrgtbccctggcccc

SEQ ID NO: 48 - DNA, 20 bp, Artificial Sequence
  FR4 B primer
ggggcmaagggaccmaggtc

SEQ ID NO: 49 - DNA, 20 bp, Artificial Sequence
  Exfor2 primer
gacctkggtcccttkgcccc

SEQ ID NO: 50 - DNA, 20 bp, Artificial Sequence
  FR4 C primer
grggsccggggacccaggtc

SEQ ID NO: 51 - DNA, 20 bp, Artificial Sequence
  Exfor3 primer
gacctgggtccccggsccyc

SEQ ID NO: 52 - DNA, 20 bp, Artificial Sequence
  FR4 D primer
ggggdcaggggacccaggtc

SEQ ID NO: 53 - DNA, 20 bp, Artificial Sequence
  Exfor4 primer
gacctgggtcccctghcccc

SEQ ID NO: 54 - DNA, 20 bp, Artificial Sequence
  FR4 E primer
acggccaggggacccaggtc

SEQ ID NO: 55 - DNA, 20 bp, Artificial Sequence
  Exfor5 primer
gacctgggtcccctggccgt

SEQ ID NO: 56 - protein, 6 aa, Artificial Sequence
  FR4 Forward primer amino acid sequence
GQGTQV

SEQ ID NO: 57 - DNA, 53 bp, Artificial Sequence
  Exback1sfi primer
gtcctcgcaactgcggcccagccggccatggccgacacggccgbctattactg

SEQ ID NO: 58 - DNA, 53 bp, Artificial Sequence
  Exback2sfi primer
gtcctcgcaactgcggcccagccggccatggccgacacggccgtttatwactg

SEQ ID NO: 59 - DNA, 53 bp, Artificial Sequence
  Exback3sfi primer
gtcctcgcaactgcggcccagccggccatggccgacacggccgtgtattaytg

SEQ ID NO: 60 - DNA, 53 bp, Artificial Sequence
  Exback4sfi primer
gtcctcgcaactgcggcccagccggccatggccgacacggccgtctattwttg

SEQ ID NO: 61 - DNA, 53 bp, Artificial Sequence
  Exback5sfi primer
gtcctcgcaactgcggcccagccggccatggccgacacggccgwttattattg

SEQ ID NO: 62 - DNA, 53 bp, Artificial Sequence
  Exback6sfi primer
gtcctcgcaactgcggcccagccggccatggccgacacggccatytattwctg

SEQ ID NO: 63 - DNA, 53 bp, Artificial Sequence
  Exback7sfi primer
gtcctcgcaactgcggcccagccggccatggccgacacgggactytattactg

SEQ ID NO: 64 - DNA, 56 bp, Artificial Sequence
  Exfor1not primer
gagtcattctcgacttgcggccgctgaaccgcctccgacctgrgtbccctggcccc

SEQ ID NO: 65 - DNA, 56 bp, Artificial Sequence
  Exfor2not primer
gagtcattctcgacttgcggccgctgaaccgcctccgacctkggtcccttkgcccc

SEQ ID NO: 66 - DNA, 56 bp, Artificial Sequence
  Exfor3not primer
gagtcattctcgacttgcggccgctgaaccgcctccgacctgggtccccggsccyc

SEQ ID NO: 67 - DNA, 56 bp, Artificial Sequence
  Exfor4not primer
gagtcattctcgacttgcggccgctgaaccgcctccgacctgggtcccctghcccc

SEQ ID NO: 68 - DNA, 56 bp, Artificial Sequence
  Exfor5not primer
gagtcattctcgacttgcggccgctgaaccgcctccgacctgggtcccctggccgt

SEQ ID NO: 69 - DNA, 57 bp, Artificial Sequence
  Exfor1cysWnot primer
gagtcattctcgacttgcggccgctgaaccgcctccgacctgrgtbccctggcacca

SEQ ID NO: 70 - DNA, 57 bp, Artificial Sequence
  Exfor1cysRnot primer
gagtcattctcgacttgcggccgctgaaccgcctccgacctgrgtbccctggcacct

SEQ ID NO: 71 - DNA, 57 bp, Artificial Sequence
  Exfor2cysWnot primer
gagtcattctcgacttgcggccgctgaaccgcctccgacctkggtcccttkgcacca

SEQ ID NO: 72 - DNA, 57 bp, Artificial Sequence
  Exfor3cysWnot primer
gagtcattctcgacttgcggccgctgaaccgcctccgacctgggtccccggscacca

SEQ ID NO: 73 - DNA, 57 bp, Artificial Sequence
  Exfor3cysRnot primer
gagtcattctcgacttgcggccgctgaaccgcctccgacctgggtccccggscatct

SEQ ID NO: 74 - DNA, 57 bp, Artificial Sequence
  Exfor4cysWnot primer
gagtcattctcgacttgcggccgctgaaccgcctccgacctgggtcccctghcacca

SEQ ID NO: 75 - DNA, 57 bp, Artificial Sequence
  Exfor5cysWnot primer
gagtcattctcgacttgcggccgctgaaccgcctccgacctgggtcccctggcagta

SEQ ID NO: 76 - DNA, 120 bp, Artificial Sequence
  mutagenesis primer for pAX001 vector
actctcgagatcaaacgggcggccgcagaacaaaaactcatctcagaagaggatctgaatggggccgcacatcatcatcaccatcacggggccgcagaaactgttgaaagttgtttagca

SEQ ID NO: 77 - DNA, 119 bp, Artificial Sequence
  muagenesis primer for pAX001 vector
actctcgagatcaaacgggcggccgcagaacaaaaactcatctcagaagaggatctgaatggggccgcacatcatcatcaccatcacggggccgcaggtggtggctctggttccggtga

(19) United States
(12) Patent Application Publication
(10) Pub. No.: US 2005/0214857 A1
(43) Pub. Date: Sep. 29, 2005

(54) Method for displaying loops from immunoglobulin domains in different contexts

(75) Inventors: Ignace Lasters, Antwerpen (BE); Jurgen Pletinckx, Nazareth (BE); Nathalie Boutonnet, Brussels (BE); Marc Lauwereys, Haaltert (BE); Els Beirnaert, Gent (BE)

Correspondence Address: MERCHANT & GOULD PC, P.O. BOX 2903, MINNEAPOLIS, MN, 55402-0903, US

(73) Assignees: Algonomics N.V., Gent (BE); ABLYNX N.V., Gent (BE)

(21) Appl. No.: 10/494,829

(22) PCT Filed: Dec. 11, 2002
(86) PCT No.: PCT/BE02/00189

§ 371 Date: Feb. 7, 2005

(30) Foreign Application Priority Data
Dec. 11, 2001 (EP) 01870274.6

(51) Int. Cl. G01N 33/53; C07K 14/705

(52) U.S. Cl. 435/7.1; 530/350

(57) ABSTRACT

The present invention is related to an isolated polypeptide micro-scaffold displaying immunoglobulin CDR2 or CDR3 polypeptide sequences, comprising a CDR2 or CDR3 polypeptide sequence interconnecting fragments of the adjacent framework polypeptide sequences, which are arranged to form two anti-parallel β-strands. The present invention is further related to a method to search, select or screen for immunoglobulin CDR2 or CDR3 polypeptide sequences that bind to a given antigen or mixture of antigens, comprising the steps of: Creating a CDR library with the method of claim 13 from the genetic information of an individual or group of individuals; Select a CDR, which binds to said antigen or mixture of antigens.

### FIELD OF THE INVENTION

The present invention is related to a novel method for displaying loops from immunoglobulin domains in different contexts. More specifically, the present invention comprises a method that allows to search for antibodies wherein antigen binding is driven by a CDR (Complementarity-Determining Regions) loop, especially HCDR3, and to identify CDR loops that maintain, enhance or maintain to a significant extent their antigen binding capacity when grafted to another structural context, especially when said structural context is expected to conformationally restrain the beginning and the end of the loop in a way that resembles the anchoring of the CDR loops on the antibody framework residues.

### BACKGROUND OF THE INVENTION

Antibodies are composed of two chains termed light and heavy chains. The light chain contains two domains: an amino-terminal variable domain (referred as VL domain) and a carboxy-terminal constant domain (CL). The heavy chain is composed of an amino-terminal variable domain (VH) and three constant domains (CH1, CH2, CH3). The antibody binding site is located in the VL and VH domains and is made up by six hypervariable loops referred to as Complementarity-Determining Regions (CDRs). Both VL and VH regions contain three CDR loops (numbered in sequence order: CDR1, CDR2 and CDR3), which are connected to a structurally conserved β-sheet framework.

It is well known that antibodies can be raised against virtually any type of antigen. The antibody-antigen binding is generally specific for the antigen against which the antibody has been raised and is usually of high affinity. Antibodies bind to the antigen at a site, which is termed the epitope. With the advent of the hybridoma technology, it became possible to produce homogeneous populations of antibodies, termed monoclonal antibodies (MAbs), marked by a single epitope specificity. MAbs have revolutionized the drug discovery work. Repeatedly it had been shown that MAbs can prevent ligand-receptor interactions thereby inhibiting ligand mediated biological effects. For example, MAb R15.7, an antibody directed against the common β-chain of the β2 family of integrins, interferes with neutrophil (expressing β2 integrins)-endothelium (expressing the β2-integrin ligands) adherence and in animal testing its effectiveness in remedying reperfusion injury has been shown (Ma et al., 1991).

It is well known that the size of an antibody can be reduced without altering the antigen recognition. Identification of the smallest antibody fragment still capable of binding to antigen has progressed from full antibody molecules to Fab and recombinant single chain Fv fragments. Now, a further reduction to single domain binding proteins based upon immunoglobulin V_{H} and V_{H}-like domains offers exciting prospects in the development of novel immunotherapeutics and immunodiagnostics.

### STATE OF THE ART

A brief historical overview of the efforts made for designing or isolating single-domain antigen binding proteins based on immunoglobulin-domains or starting from an unrelated scaffold is given below.

The driving forces behind ongoing efforts to search for such single domain antigen binding fragments is the expectation that these molecules:
are easier to identify, handle and express as recombinant polypeptide;
have superior biophysical properties such as solubility and stability, which would
   make them superior therapeutic or diagnostics reagents;
   provide tools for generating conformationally defined peptide structures with target specificity, which exhibit potential as pharmacophores for drug design.
      Immunoglobulin VH Domain:

It is widely accepted that Ig heavy chains alone retain significant antigen binding ability in the absence of a light chain. For naturally occurring antibodies this was shown already long ago (Painter et al., 1972). There is also a lot of evidence from structural studies that the CDR3 region VH domain contributes the most to antigen binding. This is based on the findings that the HCDR3 amino acid residues provide most of the surface contact area and are crucial in the molecular interaction with the antigen (Padlan, 1994).

In the early days attempts were made to isolate VH domains by enzymatic digestion, but this approach has not been successful. Due to the progress in gene technology in the 80's, the generation of recombinant VH domains came within reach. The first attempt was made in 1989 by Ward and colleagues, who made a VH expression library from the spleens of mice immunized with hen egg-white lysozyme and keyhole-limpet haemocyanin (Ward et al., 1989). Using the polymerase chain reaction, diverse repertoires of VH genes were cloned from the spleen genomic DNA of the immunized mice. The Escherichia coli (E. coli) cells secreted VH domains thereby permitting the screening of clones expressing antigen specific fragments. Binding activities were detected against both antigens and two VH domains were characterized, which showed to have nanomolar affinities For lysozyme. The fragments which were isolated were barely soluble and difficult to produce.

Based on the structural features of Camelid heavy-chain antibodies published by Hamers and colleagues at the University of Brussels (Hamers-Casterman et al., 1993), Davies and Riechman were the first to report on the camelization of VH fragments in order to cope with the insolubility of isolated VH-domains. In the first experiments, they mutated the hydrophobic residues at position 45 and 47 of the VH into hydrophilic residues. These residues are part of the hydrophobic cluster and are essential for the association of the VL- to the VH-domain. The introduction of these mutations led to an increased solubility of the VH domain and allowed the structural analysis by NMR spectroscopy (Davies and Riechmann, 1994). Another report describes the selection of a single domain VH fragment recognizing specifically a cell surface antigen from melanoma cells. The fragment V86 was a cloning artifact or derived from an in vivo recombination event isolated from a scFv phage library containing the randomly scrambled VH and VL regions of a patient immunized with genetically-modified autologous tumor cells (Cai and Garen, 1996). The strict specificity of V86 for melanoma cells was confirmed by immunohistochemical staining tests. The effect of adding a VL domain to the selected VH was examined and it was observed that the presence of the light chain fragment resulted in loss of antigen recognition or in lower affinity.

Reiter and colleagues chose a V_{H} of a mouse monoclonal with a unique VH-VL interface as a scaffold for construction of a single-domain phage-display library (Reiter et al., 1999). The library, consisting of 4×10⁸ independent clones, was generated by the randomization of nine amino acid residues in HCDR3. From these libraries specific binding clones for protein antigens were rescued Monomeric VH proteins were subsequently prepared in E. coli starting from inclusion bodies. Binding studies demonstrated an affinity of 20 nM.

VL Domain Derived Libraries

Recently it was suggested that it is feasible to isolate specific single-domain VL domains against diverse targets as previously done for single-domains VH. From a VL library derived from human B-cells and which was further diversified in its CDR regions and subjected to gene shuffling, several specific fragments for B7.1, B7.2 or human IgG were obtained (van den Beucken et al., 2001).

CTLA4 Domain

The Cytotoxic T-lymphocyte Associated antigen-4 is an important immunomodulatory protein expressed on the surface of T-lymphocytes. It binds to co-receptors B7.1 and B7.2. It is a 44 kDa homodimer, with each monomeric unit consisting of an extracellular variable domain joined via a stalk polypeptide in the membrane and an intracellular SH-2 binding domain. The variable domain consists of eight β-strands and three CDR3-like loop structures and has two disulfide bonds to stabilize the structure. Hufton and colleagues used the extracellular domain of CTLA-4 as a single immunoglobulin fold-based scaffold for the generation of novel binding ligands (Hufton et al., 2000). In their approach a phage display library was created by replacing the nine amino acid CDR3-like loop of CTLA-4 with the sequence XXX-RGD-XXX (where X represents any amino acid). Using phage display several CTLA-4-based variants capable of binding to human alphavbeta3 integrin were retrieved.

Minibody or Minimized β-Pleated Proteins

The minibody is an engineered version of a VH domain. In this molecule three strands were removed resulting in a 61 residue polypeptide consisting of a beta-pleated framework and only two hypervariable regions (CDR1 and CDR2). A library of 50 million minibodies was constructed and displayed on phage. From these libraries variants were isolated which inhibit human interleukin-6 in in vitro assays. From a selected set of minibodies competitive inhibitors for the protease encoded by the gene of the non-structural protein type 3 (NS3) from the hepatitis C virus were obtained as well (Vaughan and Sollazzo, 2001).

Shark derived New Antigen Receptor

The new antigen receptor (NAR) from nurse and wobbegong sharks has been characterized and it was demonstrated that these receptors are dimers, each chain composed of one variable and five constant domains (Roux et al., 1998). No light chain or any other protein can be demonstrated to associate with this dimer. The NAR V-region conforms to the prototype of the immunoglobulin variable domain with the canonical disulfide bridge and three CDRs. This was demonstrated by sequencing both genomic DNA and cDNA clones. At the primary sequence level a high homology with mammalian VH was observed.

To determine whether these NARs function as antigen-binding proteins, NAR was used as scaffold for the construction of protein libraries in which part of the CDR3 loop was randomized. The synthetic library was efficiently expressed on the surface of fd bacteriophage. Panning allowed the isolation of NAR proteins specific for Gingipain K protease from Porphyromonas gingivalis. Recently, Nuttall and colleagues demonstrated the involvement of these receptors in the immune response and hypothesized that these function as an antibody-like molecule (Nuttall et al., 2001). This was concluded from the finding that antigen-specific NAR-fragments were isolated out of the natural repertoire. Hudson's group is currently testing the immunization of sharks and subsequently will try to isolate binding molecules by ribosome display technology (Nuttall et al., 2002). In addition they provided evidence that NARs can be produced as monomeric fragments in E. coli and that these appear to be quite soluble, well folded and rather stable.

Further Size Reduction

The work of Kabat and Wu (Kabat and Wu, 1991) showed that within the VH domain HCDR3 plays a key role in determining antibody specificity. This observation is corroborated by structural studies where it is seen that invariably HCDR3 is involved in antigen binding and in general contributes most of the antigen contact surface area.

This crucial role of HCDR3 parallels the peculiar genetic mechanisms that give raise to HCDR3. HCDR3 originates from the rearrangement of V, D, and J region sequence elements during lymphocyte maturation. Variations in the particular V, D, and J elements used, the precise location of points of recombination, and some random nucleotide addition are all elements that contribute to the extensive length and sequence heterogeneity of HCDR3. From the work of Marks and colleagues (Marks et al., 1991) the size of the human HCDR3 repertoire, not accounting the diversity increase due to somatic mutations, was estimated to consist of about 2.3 ×10⁸ sequences. According to the work of Decker and colleagues (Decker et al., 1991) it has been predicted that the size of the mouse HCDR3 repertoire or a specific VH gene rearranged to a specific J-minigene is at least 10⁴.

While HCDR3 is playing a crucial role in antigen binding, this does not imply that the other CDRs have an insignificant role in antigen binding. Indeed, in general antigen binding is governed by interactions involving multiple CDRs (Mian et al., 1991). However a number of cases have been reported where HCDR3 peptides show antigen binding mimicking the parental antibody. For example HCDR3 of PAC1, a murine Mab binding to the GPIIb-IIIa platelet fibrinogen receptor, mimics PAC1 in inhibiting (Kᵢ=10 μM) fibrinogen mediated platelet aggregation (Taub et al., 1989). More recently, it was shown that HCDR3 of IgGl b12 (Saphire et al., 2001) is capable of neutralizing HIV-l variants albeit at an apparently higher IC50 as compared to the IgG1 b12 from which this HCDR3 was derived. This shows that at least some HCDR3 loop regions have the potential to be used in constructs that differ from the parental antibody. A nice example of such construct was provided by Smith and colleagues (Smith et al., 1995) who showed that HCDR3 from Fab-9, an antibody binding the beta 3-integrins with nanomolar affinity, could be grafted into the epidermal growth factor-like module of human t-PA resulting in a variant binding to platelet integrin with nanomolar affinity.

That it may be useful to constrain somehow HCDR3 to mimic the parental antibody binding was recently shown for a 19 amino acids cyclic peptide comprising HCDR3 of mAB63 (Deng and Notkins, 2000). This cyclic peptide showed the same antigen binding pattern as the parental mAb63.

There are some important shortcomings in the prior art at several levels.

Firstly, the antibody size reduction is arrested at the level of small protein domains (VH, minibodies, etc). Such small protein domains may be endowed with desirable properties, especially the VHH domains derived from Camelid antibodies which are highly soluble and can bind with high affinity to a given antigen and do not cross-react with non-related antigen (Arbabi Ghahroudi et al., 1997). However, as these smaller constructs should still be considered as protein entities it is far from straightforward to further reduce their size such that the resulting constructs become amenable for the design or identification of small molecule analogues mimicking the binding of the larger construct. Hence, strategies to further reduce the antibodies size are needed.

Secondly, while it is tempting to use HCDR3 from VH or VHH domains as a next step for the size reduction, in view of their strong involvement in antigen binding (and perhaps especially so in the VHH scaffold wherein often HCDR3 loop is quite long and can penetrate cavity regions, e.g. the active site of an enzyme such as carbonic anhydrase (Desmyter et al., 2001), only a limited number of cases have been reported wherein a HCDR3 peptide shows binding affinity mimicking the binding of the parental antibody construct. As a consequence there is a need for techniques that allow to effectively and efficiently search or screen for cases wherein isolated CDR3 loops, especially HCDR3 loops, show significant binding to a given antigen of interest.

Thirdly, there is need for rational strategies for size reduction such that structural information on the resulting construct, e.g. a HCDR3 loop region, can be obtained. Such structural information is important to initiate a further reduction leading to small molecule mimicking the binding of the original antibody construct to at least a significant extent, meaning that the binding is competitive with respect to the original antibody and is statistically significantly above the background signal as measured by assay systems, such as an ELISA system, as is known by a person skilled in the assessment of binding affinity.

### AIMS OF THE INVENTION

The present invention aims to provide a method to identify, search or select peptides, preferably HCDR3 peptides, that bind to a given target or targets of interest. The method intends also to graft the found peptides to a suitable protein scaffold, immunoglobulin or other protein scaffold. This grafting may be advantageous if said scaffold is endowed with useful properties relating e.g. to targeting, solubility or stability.

### SUMMARY OF THE INVENTION

The present invention concerns in a first aspect an isolated polypeptide micro-scaffold displaying immunoglobulin CDR2 or CDR3 polypeptide sequences, comprising a CDR2 or CDR3 polypeptide sequence interconnecting fragments of the adjacent framework polypeptide sequences, which are arranged to form two anti-parallel β-strands. Preferably, the CDR3 polypeptide sequences are HCDR3 polypeptide sequences.

The micro-scaffold of the present invention preferably has said framework polypeptide sequences selected from the group consisting of naturally occurring immunoglobulin framework polypeptide sequences, mutated naturally occurring framework polypeptide sequences, and artificial consensus framework polypeptide sequences. In a preferred embodiment, said framework polypeptide sequences is a mutated naturally occurring framework polypeptide sequences comprising cysteine residues at Kabat numbering positions 92 and 104 arranged to form a disulphide bridge crosslink for increasing the conformational stability of the anti-parallel β-strands.

The micro-scaffold according to the invention can be linked to a polypeptide suitable for presenting or expression of said micro-scaffold.

Further, said polypeptide suitable for presenting or expression preferably is a surface protein of a viral system with a solvent accessible N-terminus or C-terminus.

Another aspect of the present invention concerns an isolated nucleotide sequence encoding the polypeptide micro-scaffold of the present invention.

A further embodiment of the present invention is a vector comprising the isolated nucleotide sequence as mentioned above.

In yet another aspect of the present invention, a CDR polypeptide library of micro-scaffolds according to the present invention is disclosed, characterised in that the CDR2 or CDR3 polypeptide sequences of a sufficient number of micro-scaffolds represent at least a significant fraction of a natural repertoire. The CDR polypeptide library of the present invention preferably has said sufficient number of micro-scaffolds lies between 10 and 10¹⁵.

In another aspect of the present invention, a CDR nucleic acid library of micro-scaffold nucleotide sequences according to the present invention is disclosed, characterised in that the CDR2 or CDR3 nucleotide sequences of a sufficient number of micro-scaffolds represent at least a fraction of a natural repertoire.

In yet another aspect of the present invention, a method for creating a micro-scaffold according to the present invention, comprising the steps of:
Providing a CDR2 or CDR3 nucleotide sequence interconnecting fragments of its adjacent framework nucleotide sequences to obtain a micro-scaffold nucleotide sequence, and
Express said micro-scaffold nucleotide sequence in a suitable system.

A further aspect of the present invention concerns a method for creating a CDR library displaying loops of immunoglobulin domains, comprising the steps of:
Prepare a CDR nucleic acid library as described above, and
Express said CDR nucleic acid library in a suitable system.

The method is preferably further characterized in that said suitable system is a viral system having a surface protein with a solvent accessible N-terminus or C-terminus.

A further aspect of the present invention concerns a method to search, select or screen for immunoglobulin CDR2 or CDR3 polypeptide sequences that bind to a given antigen or mixture of antigens, comprising the steps of:
Creating a CDR library with the method of claim 13 from the genetic information of an individual or group of individuals,
Select a CDR which binds to said antigen or mixture of antigens.

A further aspect of the present invention concerns a method to search, select or screen for immunoglobulin CDR2 or CDR3 polypeptide sequences that bind to a given antigen or mixture of antigens, comprising the steps of:
Creating a CDR library with the method of the invention from the genetic information of an individual or group of individuals,
Creating a VH, Fab, scFv or IgG library from the genetic information of said individual or said group of individuals, and
Selecting a CDR which binds to said antigen or mixture of antigens, in both said CDR library and said VH, Fab, scFv or IgG library.

Yet another aspect of the present invention concerns a method to search, select or screen for immunoglobulin CDR2 or CDR3 polypeptide sequences that bind to a given antigen or mixture of antigens, comprising the steps of:
Creating a CDR library with the method of the invention from the genetic information of an individual or group of individuals,
Creating a non-immunoglobulin grafted CDR library using a non-immunoglobulin scaffold that is arranged arranged to comprise grafted CDR loops of said CDR library, and
Selecting a CDR which binds to said antigen or mixture of antigens, in both said CDR library and said non-immunoglobulin grafted CDR library.

In the method of the invention, wherein the individual or group of individuals can be either immunized or naïve to the given antigen or mixture of antigens.

Another aspect of the present invention concerns a method for designing, selecting or screening peptide molecules, with a sequence homologous or relative to the sequence of the CDR sequences identified by the method of any of the claims 15 to 18, said sequence binding to the antigen or mixture of antigens used.

### SHORT DESCRIPTION OF THE DRAWINGS

FIGS. 1a and b both represent a micro-scaffold according to the present invention.

FIG. 2 is a schematic representation of the amplification and cloning strategies for obtaining the human naïve VH and HCDR3 microscaffold (VH_{μs}) libraries.

FIG. 3 shows the analysis on agarose gel of primary PCR products coding for the naïve human VH gene products.

FIG. 4 shows purified PCR products coding for the human VH after a second amplification analysed on 1.5% low-melting agarose.

FIG. 5 shows the analysis on agarose gel of primary PCR products coding for the VHH gene products from the immunized llama

FIG. 6 shows the analysis on agarose gel of the HCDR3-sequences amplified from the dedicated VHH-library.

FIG. 7 represents the pAX001 vector.

FIG. 8 represents the fdtet phage.

FIG. 9 shows a western blot analysis of the gene3 fusion products of 8 different (llama VHH derived) HCDR3 clones.

FIG. 10 represents a phage ELISA test with polyclonal phage from non-selected libraries on IL-6, IGE and the negative control (β-casein).

FIG. 11 shows the enrichment after one round of selection on THF and CEA as visualized by the number of transfected E. coli colonies on agar plates.

FIG. 12 shows the length distribution of HCDR3 in the non-selected immune library derived from llama.

### DETAILED DESCRIPTION OF THE INVENTION

CDR Libraries

In view of observation that only occasionally isolated CDR loops, especially HCDR3 loops, have been found to bind towards a given target of interest, one needs a strategy to search for such cases starting from a repertoire of candidate CDR loops. In addition one should preferably restrain conformationally these CDR loops to mimic, at least to some extent, the loop anchoring on framework residues as observed in natural antibodies. There are at least two additional lines of thought which provide interesting elements motivating the use of HCDR3 as a source of biologically active peptides:

Firstly, there is accumulating evidence that in the process of B cell maturation a selection of HCDR3 sequence patterns occurs. For example, the HCDR3 of pre B cell frequently contain a consecutive stretch of hydrophobic residues, which appears to be rarely seen in mature B cells. On this basis it was hypothesized (Raaphorst et al., 1997) that structural limitations by the antigen binding site promote hydrophylic HCDR3 sequences via a process of positive selection. Clearly, one should not view the repertoire of HCDR3 loops as just a source of random peptides (in length and in sequence). A random repertoire would be of limited use in view of its tremendous undersampling giving the finite repertoire size. Rather a HCDR3 peptide library should be viewed as a “biologically filtered” random peptide library. The undersampling is less of an issue as many biologically irrelevant sequences have been filtered out in the course of repertoire generation.

Secondly, it is known for some time that natural (pre-immune) antibodies have polyreactive phenotype and this polyreactivity (i.e. being able to recognize multiple epitopes) can be attributed to HCDR3 (Chen et al., 1991). Most interestingly, in a study on Fab fragments retrieved from combinatorial IgG libraries prepared from the bone marrow of long term asymptomatic HIV seropositive donors (Ditzel et al., 1996) it was shown that a constrained peptide based on a HCDR3 sequence was polyreactive and could inhibit the binding of the parental antibody to a panel of different antigens. The authors suggest that polyreactivity is associated with the conformational flexibility of HCDR3. This view is supported by a study on the effect of amino acid substitutions in HCDR3 of an auto-antibody on its polyreactivity (Adib-Conque et al., 1998). Substituting prolines into glycines in HCDR3 (expected to augment the loop's plasticity) resulted in Fab fragments that were highly polyreactive. Together these findings suggest that HCDR3 libraries, including naive HCDR3 libraries, may be a particularly rich source of binding structures and therefore may be ideally suited to screen for peptide drug leads.

### DEFINITIONS

All technical or scientific terms used herein have the same meaning as known or understood by someone skilled in the art of molecular genetics, nucleic acid handling, cloning, phage display, Polymerase Chain Reactions (PCR) and biochemistry.

Standard techniques are always used to carry out the individual steps of the present invention as can be found in standard in e.g. PCR Protocols: A guide to Methods and Applications (Innis et al., 1990. Academic Press, San Diego, Calif.), Phage Display of Peptides and Proteins, A laboratory Manual (Brian K. Kay et al., 1996, Academic Press, San Diego, Calif.) . These references are quoted here solely to illustrate that good reference books are readily available to document in detail all standard procedures.

In the context of the present invention a number of definitions are specified. A repertoire is meant to be a collection of different entities, each represented by a certain copy-number (designating the number of times the given entity occurs in the repertoire). These entities correspond generally to nucleic acid sequences, each of which in part or in whole encodes a peptide or polypeptide. The term repertoire denotes a collection of entities that exists in nature, such as e.g. the immunoglobulin repertoire of humans. The term library denotes a collection of entities obtained via molecular genetics or other means from a given repertoire of entities. The size of the repertoire or of the library corresponds to the number of different entities it contains. When the library is physically implemented in e.g. a suitable viral system such the M13 phage or phagemid systems, the size is often expressed in the number of so called unique clones, abbreviated as u.c.

In the context of the present invention the libraries will be derived from nucleic acid sequences encoding the whole or parts of antibodies, preferably the variable domains (comprising the complementary determining regions also denoted as CDR regions).

The term “starting library” refers to the library of nucleic acid sequences, prior to exploring the library. By exploring is meant that the library is handled in such a way that (a) the peptide or polypeptide sequences encoded by each of the nucleic acid sequences held in the library are displayed on a vehicle that contains in its genetic material said nucleic acid sequence, (b) these vehicles are presented at some concentration to some target of interest for a certain time at given conditions of pH, ionic strength, temperature and pressure, (c) the bound vehicles are subsequently obtained by washing away the vehicles that are not bound to the target and subsequent eluting the bound vehicles by e.g. acid or other treatment, (d) the retrieved vehicles are subsequently propagated or amplified such that enough vehicles are produced to repeat the whole process, referred to as biopannning, starting from (b).

To overcome the shortcomings of the prior art the following procedure should be preferably followed. The procedure may be altered, or further optimized following state of art insights familiar to molecular biologists and/or biochemists. An essential step of the present invention is that the size reduction is achieved by a screening or selection process using a starting library of candidate constructs. The starting library, should contain between 10 and 10¹² candidate elements. Often, for practical reasons, the library size does not exceed 10⁶, 10⁷, 10⁸ or 10⁹ candidate elements. Such libraries are also considered as valuable and preferable. Ideally, the library should contain as many as possible constructs as someone familiar with the art of library generation is capable to make following state of the art techniques. In order to handle in practice a library, the library should contain all the genetic information needed to express the encoded polypeptides defined by the elements of the library. Typically, the library corresponds to a collection of different DNA segments (encoding the peptides or proteins of the library), each of which is engineered (as can be done by any molecular biologist familiar with the state of art in the field of genetic engineering) in a vector of interest, be it a phagemid, phage, chromosome or other vehicle.

Preferably, but not mandatory, these constructs will entail the HCDR3 regions of the heavy chain variable domains of a repertoire of antibodies derived by standard techniques, known by someone familiar with antibody engineering. Equally preferable, these constructs will entail the light chain CDR3 (LCDR3) regions of the light chain variable domains of a repertoire of antibodies. Less preferred are the regions corresponding to other loop regions in the variable domains of the heavy or light chains of a repertoire of antibodies.

Libraries of antibodies or of antibody domains (such as VH of VHH in the Camelid antibodies), referred to below as parental libraries, are obtained either from non-immunized individuals (one or more humans or animals), such parental libraries being denoted as naïve parental libraries, or from immunized individuals (one or more humans or animals) against one or more targets of interest, such parental libraries being denoted as dedicated parental libraries. The interest in starting from naïve parental libraries should not be under-appreciated and is motivated as follows. Firstly, it is not unlikely that a dedicated parental library may disfavor to some extent antibodies where antigen binding is fully driven by HCDR3. This may occur if in the process of affinity maturation the interaction with the antigen is optimized via additional contributions provided by the other CDRs or by some framework residues. Clearly, this would lead to a situation where sub-optimal binders tend to be eliminated from the dedicated library. However, such binders are very valuable as these may yield new peptide drug leads that may be further optimized by e.g. spiked randomization of the retrieved CDR3 loop motifs. Secondly, working with a naive parental library is advantageous in the sense that it avoids repeated immunizations and library constructions. This is of particular importance when the antigen would represent for instance a biological hazard or toxic agent, which would raise complex safety issues with respect to the immunization of animals or humans.

In the next step of the process and starting from a naïve or from a dedicated antibody or antibody domain repertoire, one produces a library of CDR loops, preferably HCDR3 loops, wherein in each loop is anchored on an adjacent segments of residues to anchor the loop region and such that the base of the loop region gets conformationally constrained, i.e. it has reduced conformational freedom as compared to an isolated CDR loop. As these segments can be viewed as a scaffold to anchor the loop, these segments together with the CDR loop are denoted below as micro-scaffold. Hence, starting from a naive parental library, one obtains a naïve micro-scaffold library and similarly starting from a dedicated parental library a dedicated micro-scaffold library is obtained. To engineer the micro-scaffold library one can follow state-of-the art techniques employing PCR steps with one ore more primers or sets of primers to amplify the CDR loops from a pool of DNA molecules obtained from the proper parental library (naïve or dedicated parental library) in the context of the preceding and succeeding antibody framework residues. More specifically, it is preferable that the extension sites of these primers match with nucleotides at or near the end of the regions preceding and succeeding the CDR loop. This is because in general the sequence variability in the loops is considerably much larger than in the surrounding framework residues and consequently in order to amplify as much as possible the CDR library, the primers or set of primers should best be designed to match in the more conserved framework residues adjacent to the CDR. It is also useful to flank these primers with suitable restriction sequences for subsequent efficient cloning in any suitable vector of interest, be it a phagemid, phage or other vector.

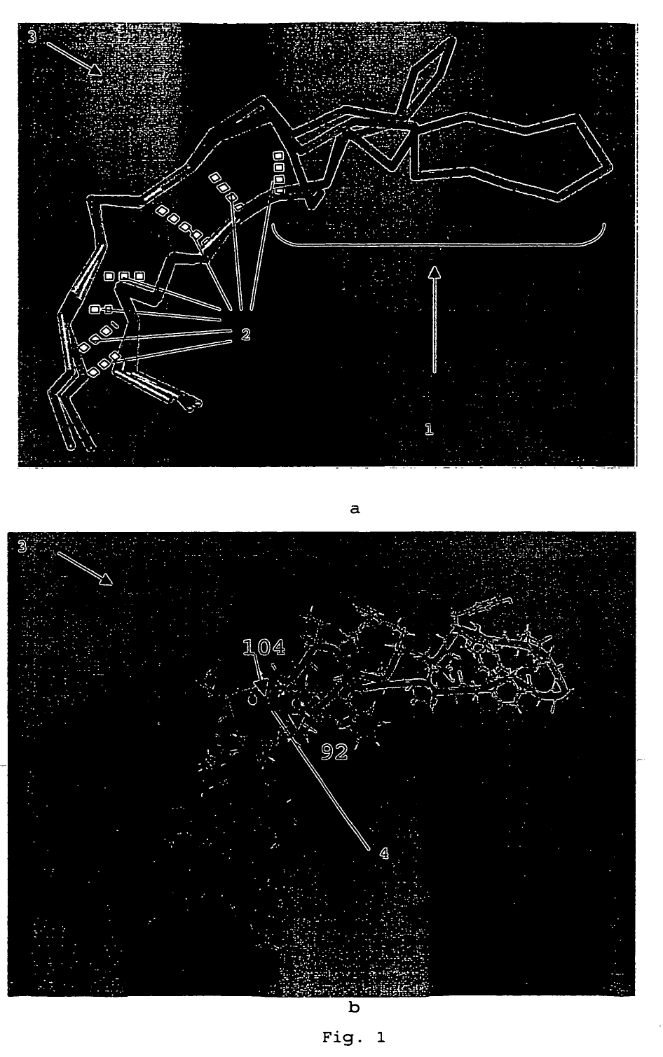

In the micro-scaffold approach addressing HCDR3 loops, the loops will be displayed in the micro-scaffold which is meant to conformationally restrain the base of the loop. In VH or VHH the HCDR3 loop is anchored on FR3 and FR4. As these regions are extended structures implied in an anti-parallel beta sheet organization at least the last part of FR3 and FR4 (the end of the variable heavy chain domain) are included in the micro-scaffold, thereby intending to constrain the base of the HCDR3 loop much as in the parental construct. The typical layout of the micro-scaffold is shown in FIG. 1.

The micro-scaffold library should preferably have a similar size as the associated parental library but may, in view of practical considerations, also be of a size smaller or even be considerably smaller as the parental library. In order to provide enough anchoring to the CDR loop, the adjacent framework segments should be at least two, preferably 3, 4, 5 or even up to 10 or more residues in length. Preferably, the process intends to rescue the HCDR3 library expressed in the micro-scaffold context encompassing the end of framework region FR3, typically residues 86 until 92 (using standard Kabat numbering) and the whole or most of the FR4 framework region.

In a third, optional step of the process that specifically applies to HCDR3 derived micro-scaffold libraries, the base of the HCDR3 loop may further be conformationally restrained by the introduction of a non-natural disulfide bridge. Specifically, by substituting Gly 104 (Kabat numbering) into Cys in FR4, a non-natural disulfide bridge can be introduced with the conserved Cys 92 (Kabat numbering) at the end of FR3. In the micro-scaffold context, it is expected, in view of the proximity of both residues, that a disulfide bridge is likely to be made, thereby strengthening the base of HCDR3. To engineer this Gly104Cys substitution, one can typically, either starting from the library in step 2 or the library of step 1, reinforce the substitution in the PCR amplification process using appropriate forward primers (matching in FR4) and using a backward primer (or set of backward primers) matching in FR3. Typically, one will work with one or with a mixture of forward primers wherein the Gly codon is switched into Cys by one nucleotide substitution (G to T substitution in the first nucleotide of the codon). Preferably, this substitution should be located at two, three, four or more nucleotides from the 3′ extension point of the forward primer(s). Usually this substitution will be introduced starting from the micro-scaffold library of the previous step. Alternatively, the forward primers used to generate the micro-scaffold library of the previous step may already carry the required substitution forcing the substitution into Cys at position 104.

Subsequently, the micro-scaffold library, produced by step 2 or by step 3 (wherein a Cys was introduced at position 104 of FR4), is expressed by applying standard techniques in such a way that the micro-scaffold encoded DNA is expressed as an polypeptide or as a polypeptide that is linked to another protein. Typically, the micro-scaffold will be anchored via an optional linker to the N-terminus of the minor coat protein pIII of the M13 phage enabling the display of the micro-scaffold library on phage or on phagemid particles. The advantage of this procedure is that the micro-scaffold library becomes then displayed on a vehicle that contains the necessary genetic material encoding the displayed polypeptide, thereby allowing to search/select for binding peptides in an iterative manner via standard phage display techniques known by anyone who is familiar with the techniques of phage display. Typically, two or three (and more rarely four or more) rounds of so-called biopanning are done with the micro-scaffold library against the target of interest. In the case a dedicated micro-scaffold library is used it is natural, but not mandatory, to biopan said library against one or more targets that were used in the immunization step prior to rescuing the antibody repertoire response.

Concomitantly with this step, the parental library (naïve or dedicated) will preferably be explored via the same or a similar protocol in biopanning against the same target of interest.

Finally, the retrieved binders obtained in the course of biopanning with the micro-scaffold library (after each of the rounds of biopanning) can be characterized by phage ELISA or similar techniques (that are familiar by anyone working in the field of phage display). The genetic material obtained from a set of binding clones is subject to sequence analysis (which can at present be done using fully generic techniques) to determine the sequence of at least the CDR region of the micro-scaffold. It is customary to sequence a reasonable amount of clones (5, 10 or more clones) in order to identify in a sound statistical way which residues in the retrieved sequence patterns are likely to contribute to the antigen binding. Preferably, but not mandatory, the same analysis is done on the resulting binders of the biopanning with the parental library against the same target. The advantage is that by comparing the sequences retrieved from biopanning with both libraries (micro-scaffold and parental libraries) it is possible to identify which sequence patterns are common to both libraries, in order words it becomes possible to indicate which CDR loops bind to the given target in both structural contexts (parental construct and micro-scaffold construct). In case no biopanning is done with the parental library, such comparison cannot be done. However, in such case the procedure remains advantageous and valuable for at least two reasons.

Firstly, peptide leads can be identified as a result of the biopanning procedure. As the peptides are conformationally constrained by the micro-scaffold, the peptides are presented in a less flexible way and this may well increase the likelihood to identify binding peptides especially directed against cavities on the surface of the antigen.

Secondly, even one is not interested in peptide leads but only in protein-based therapeutic or diagnostic agents, the usage of the micro-scaffold library is advantageous. Indeed, as the found binding peptides (HCDR3 peptides in case the micro-scaffold corresponds to a HCDR3 naïve or dedicated library) have been explored in a constrained way (fixing or restraining the base of the loop), it becomes well feasible to graft the found peptides into a scaffold that would (a) expose the loop towards the solvent and (b) restrains the loop in similar way as in the micro-scaffold. Clearly, in view of the design of the micro-scaffold, the retrieved binding peptides are likely to be grafted on a antibody variable domain (VHH or VH in case the loop corresponds to a HCDR3 loop), thereby conserving its binding towards the antibody. As a result the obtained construct (variable antibody domain with grafted binding loop) can be further used as a therapeutic or diagnostic agent. This procedure may become particularly attractive and become a generic procedure if the domain onto which the loop is grafted has first been de-immunized to ensure that it does not contain any T cell epitopes.

Thirdly, the found peptides can be grafted on a scaffold of known 3D structure that has anchoring positions for the loop that are compatible with the micro-scaffold structure. Clearly, antibody domains are ideal candidates for this as many structures are known and in view of the design of the micro-scaffold, the loop can be grafted on framework residues that are encompassed in the micro-scaffold definition. But also, other proteins can be used, such as BPTI (bovine pancreatic trypsin inhibitor) that contain anti-parallel beta-strands organized in a sheet. In this case the loop can be inserted as a connecting loop between the beta-strand of the sheet. Following the grafting the binding capacity against the original antigen should be assayed. If binding is confirmed, the loop conformation may be identified via X-ray crystallography of the protein or protein domain on which the loop was grafted. Clearly, based on the obtained structural information, peptidometric research can be initiated to design small molecules mimicking the loop conformation.

FIGS. 1a and b both represent a micro-scaffold according to the present invention. This figure shows two HCDR3 loops taken out of two different structurally known VH domains, anchored on the FR3 and FR4 regions that where truncated to match the design of the micro-scaffold. It is clearly seen, that the anti-parallel beta sheet is well preserved and that, as expected, the structural variability fully resides in the HCDR3 loop (1). The dashed lines in FIG. 1 a highlight the hydrogen binding network 2 in the micro-scaffold. FIG. 1b shows a detailed look of a particular example of a HCDR3 loop anchored on the micro-scaffold 3. This picture illustrates that the base of the HCDR3 loop can be further restrained by engineering a non-natural disulfide bridge (4) between framework residues at the end of FR3 and the beginning of FR4. One preferably engineers this disulfide bridge between position 92 (Kabat numbering), a conserved Cys residue and position 104 (Kabat numbering) a conserved Gly residue. Other sites to introduce a disulfide bridge might also be considered.

### EXPERIMENTAL EXAMPLES

### Example 1

### Construction of Human Naïve VH and VH Micro-Scaffold ( VH_{μs}) Libraries

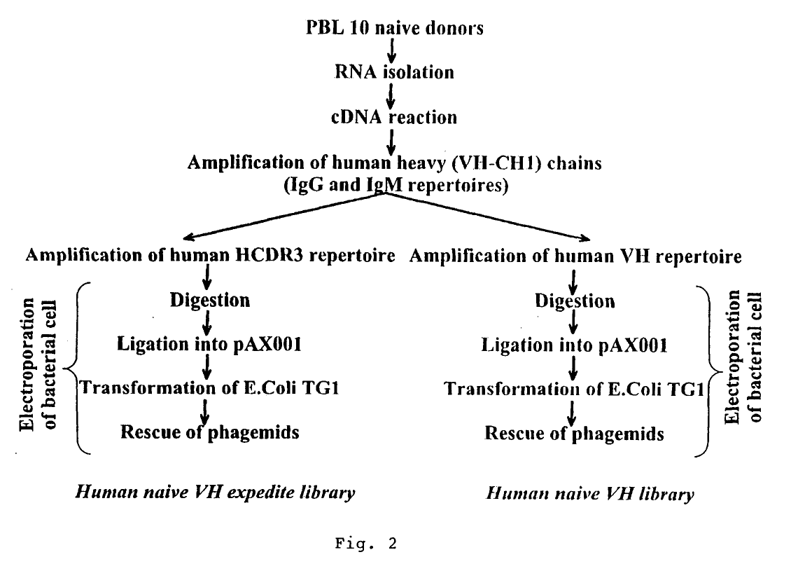

The naive VH and VH_{μs} libraries are built in parallel, following the procedure shown in FIG. 2. The first three steps (RNA isolation, cDNA reaction and amplification of human heavy chains) are common for both libraries. The obtained PCR fragments of the primary amplification of the heavy chains are then used as template for the construction of the VH and VH_{μs} libraries.

RNA Isolation

mRNA from peripheral blood lymphocytes (PBL) from 10 healthy donors was extracted as described by Chomczynski et al., 1987. Briefly, after isolation of the PBL on a Ficoll-Hypacque gradient, the cell pellet was dissolved in 8 M guanidinium thiocyanate, 25 mM citric acid, 17 mM N-lauroyl sarcosine and 0.1 M β-mercapto-ethanol. Chromosomal DNA was sheared by passing through a 19 Gauge needle and a 23 Gauge needle. Next, two phenol-chloroform extractions followed by two ethanol precipitations were performed. RNA was resuspended in 70% ethanol and 20 mM sodium acetate pH4.0 and stored at −80° C. The total yield of RNA for the 10 donors varied between 300 μg to 950 μg as determined by OD_{260:28 nm} measurement. 5 μg mRNA was treated with 1 M glyoxal, 50% DMSO, 10 mM NaH₂PO₄ (pH7) for 1 hr at 50° C. and analysed on a 1% agarose gel. The gel was stained in 10 μg/ml ethidiumbromide in 50 mM NaOH for 30 min and destained in 0.5 M Tris-HCl (pH7.5) for 30 min.

cDNA Reaction

Random primed or oligo-dT cDNA was prepared from 200 μg mRNA, by heat denaturation of RNA for 5 min at 65° C. in the presence of 10 μg oligo-dT or random primers (Amersham Pharmacia Biotech, Uppsala, Sweden). Subsequently, buffer and 10 mM DTT was added according to the manufacturers instructions (Invitrogen, Merelbeke, Belgium) together with 500 μM dNTP (Amersham Pharmacia Biotech, Uppsala, Sweden), 400 units RNAsin (Promega) and 1,000 units MMLV reverse transcriptase (Invitrogen, Merelbeke, Belgium) in a total volume of 250 μl. After 2 hrs incubation at 42° C., the cDNA was purified by means of two phenol-chloroform extractions and an ethanol precipitation and dissolved in 100 μl distilled water.

Amplification of Human Heavy Chain Variable Regions from IgG and IgM

The complete IgG and IgM genes were amplified with olio-dT primer combined with family specific VH-Back primers (Table 1) on oligo-dT primed cDNA as template according to the methods as described in EP01205100.9. The IgG amplicons (1.6 kB) and the IgM amplicons (2.1 kB) were gel purified and used as template for a secondary amplification for introduction of a SfiI-site in the Back-primers as is described in the next section.

Alternatively, primary amplification of the genes coding for the variable regions of the heavy chains was performed with two different sense primers (Hu-IgGl-CH1-For and Hu-IgM-CH1-For) to obtain the IgG and IgM repertoire. The sense primers are located in the 3′ part of the constant region of the heavy chain. Eight different antisense primers located in the 5′ part of VH (called VH-Back) were used. Highly homologous antisense primers were combined in the same reaction, resulting in five different combinations for the sense primer Hu-IgG1-CH1-For and Hu-IgM-CH1-For. The oligonucleotide primers used for the primary amplification of the heavy chain variable regions are shown in Table 1.

TABLE 1
A. PRIMARY AMPLIFICATION (5′->3′)

| | |
|---|---|
| VH1B/7A-Back | CAGRTGCAGCTGGTGCARTCTGG |
| VH1C-Back | SAGGTCCAGCTGGTRCAGTCTGG |
| VH3B-Back | SAGGTGCAGCTGGTGCAGTCTCG |
| VH5B-Back | CARGTGCAGCTGGTGCAGTCTGG |
| VH4C-Back | CAGSTGCAGCTGCAGGAGTCSGG |
| VH6A-Back | CAGGTACAGCTGCAGCAGTCAGG |
| VH2B-Back | CAGRTCACCTTGAAGGAGTCTGG |
| VH4B-Back | CAGGTGCAGCTGCAGCAGTGGGG |
| Hu-IgG1-CH1-For | GTCCACCTTGGTGTTGCTGGGCTT |
| Hu-IgM-CH1-For | TGGAAGAGGCACGTTCTTTTCTTT |

B. SECONDARY AMPLIFICATION (5′->3′)

| | |
|---|---|
| VH1B/7A-SfiI-Back | GTCCTCGCAACTGCGGCCCAGCCGGCCATGGC |
| | CCAGRTGCAGCTGGTGCARTCTGG |
| VH1C-SfiI-Back | GTCCTCGCAACTGCGGCCCAGCCGGCCATGGC |
| | CSAGGTCCAGCTGGTRCAGTCTGG |
| VH3B-SfiI-Back | GTCCTCGCAACTGCGGCCCAGCCGGCCATGGC |
| | CSAGGTGCAGCTGGTGGAGTCTGG |
| VH5B-SfiI-Back | GTCCTCGCAACTGCGGCCCAGCCGGCCATGGC |
| | CGARGTGCAGCTGGTGCAGTCTGG |
| VH4C-SfiI-Back | GTCCTCGCAACTGCGGCCCAGCCGGCCATGGC |
| | CCAGSTGCAGCTGCAGGAGTCSGG |
| VH6A-SfiI-Back | GTCCTCGCAACTGCGGCCCAGCCGGCCATGGC |
| | CCAGGTACAGCTGCAGCAGTCAGG |
| VH2B-SfiI-Back | GTCCTCGCAACTGCGGCCCAGCCGGCCATGGC |
| | CCAGRTCACCTTGAAGGAGTCTGG |
| VH4B-SfiI-Back | GTCCTCGCAACTGCGGCCCAGCCGGCCATGGC |
| | CCAGGTGCAGCTGCAGCAGTGGGG |
| JH1/2-NotI-For | GAGTCATTCTCGACTTGCGGCCGCTGAGGAGA |
| | CGGTGACCAGGGTGCC |
| JH4/5-NotI-For | GAGTCATTCTCGACTTGCGGCCGCTGAGGAGA |
| | CGGTGACCAGGGTTCC |
| JH3-NotI-Eor | GAGTCATTCTCGACTTGCGGCCGCTGAAGAGA |
| | CGGTGACCATTGTCCC |
| JH6-NotI-For | GAGTCATTCTCGACTTGCGGCCGCTGAGGAGA |
| | CGGTGACCGTGGTCCC |

The primary PCR was performed in 50 μl reaction volume using 25 pmol of each primer. 2.5 μl random primed or oligo-dT cDNA was used as template, which is the equivalent of 5 μg mRNA. The reaction conditions for the primary PCR were 11 min at 94° C., followed by 30/60/120 sec at 54/55/72 ° C for 30 cycles, and 5 min at 72° C. All reactions were performed with 2.5 mM MgCl₂, 200 μM dNTP (Roche Diagnostics, Brussels, Belgium) and 1.25 U AmpliTaq Gold DNA polymerase (Applied Biosystems, Lennik, Belgium).

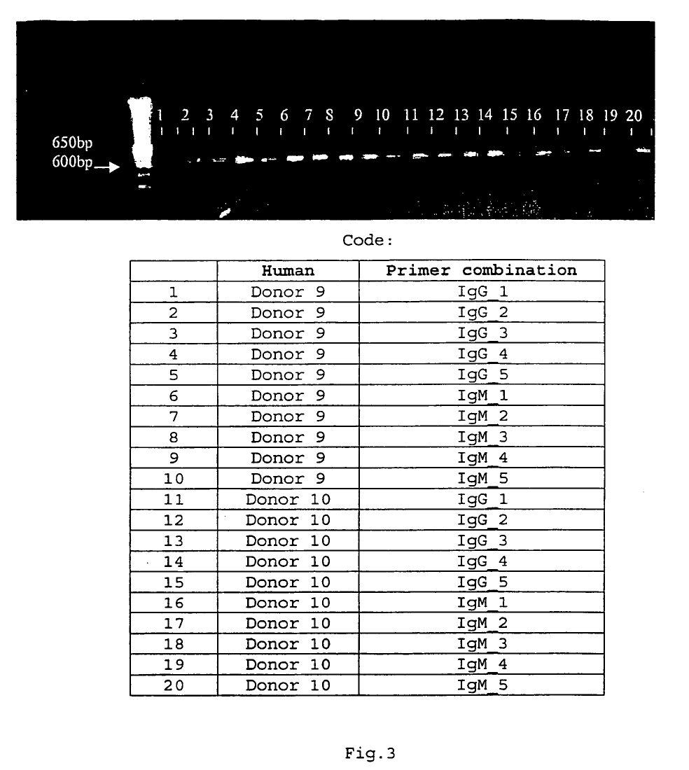

All the PCR products were separated on a 1% agarose gel and the DNA was eluted using the QIAquick gel extraction kit or QIAEXII (Qiagen, Westburg, Leusden, The Netherlands) (FIG. 3).

Construction of Human Naïve VH Library

Amplification of VH Fragments

The gel purified complete IgG- and IgM-genes were re-amplified with oligo-dT primer and extended Back-primers containing a SfiI-site. After gel purification the products were digested with SfiI and BstEII; the latter restriction enzyme cuts in most VH derived J-sequences.

Alternatively, a secondary amplification of the obtained PCR fragments of the heavy chain variable regions obtained with the CH1-primers of IgG1 or IgM was performed using four sense primers with NotI restriction sites in two combinations and 8 antisense primers with SfiI restriction sites in five combinations (Table 1). The sense primers are located in the J region while the antisense primers are located in the 5′ part of VH. The reaction was performed in 50 μl reaction volume with 25 pmol of each primer and 30 ng of purified DNA. The reaction conditions for the secondary PCR were 11 min at 94° C., followed by 30/60/120 sec at 94/55/72 ° C. for 30 cycles, and 5 min at 72° C. All reactions were performed with 2.5 mM MgCl₂, 200 μM dNTP (Roche Diagnostics, Brussels, Belgium) and 1.25 U AmpliTaq Gold DNA polymerase (Applied Biosystems, Lennik, Belgium).

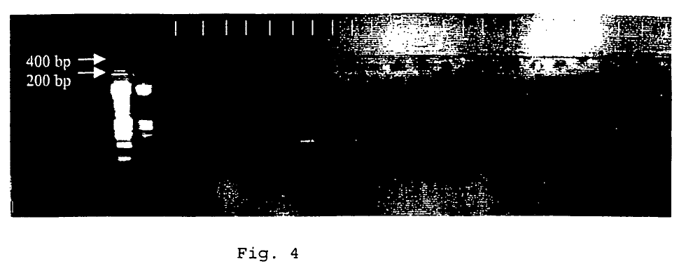

All PCR products were separated on 1.5% agarose gel. The DNA was eluted using the QIAquick gel extraction kit or QIAEXII (Qiagen, Westburg, Leusden, The Netherlands). Some of the results are shown in FIG. 4.

Electroporation of Bacterial Cells

The PCR products of the secondary amplification were digested with SfiI and BstEII or NotI in separate reactions. After desalting the digestion reactions with Microcon-YM-30 (Amicon, Beverly, Mass., USA), 500 ng of PCR fragments were ligated to 5 μg vector pAX001 linearized with SfiI and BstEII or NotI (see section Methods) using T4 DNA ligase (Promega, Leiden, The Netherlands). After desalting the ligation reactions with Microcon-YM-30, 10 μl of the ligation products were mixed with 100 μl of electrocompetent TG1 cells (see section Methods) and placed on ice. The cell/DNA mixture was transferred to 0.2 cm cuvettes (Biorad, Nazareth, Belgium) and pulsed in the Biorad Gene pulser™ (200 Ohm, 25 μFD, 2.5 kVolt, 4-5 mSec). After electroporation, 1 ml 2TY medium was added to the cuvettes and the mixture was transferred to a tube. The cells were plated on LB-agar containing 100 μg/ml ampicilin and 2% glucose using 30 cm² square petridishes. Also, dilutions (10⁻² to 10⁻⁶) were plated in 9-cm Ø petridishes to determine the size of the libraries. The library was harvested after overnight incubation at 37° C. by flooding the plates with 5-10 ml 2TY/ampicilin/glucose and detaching the cells by scraping with a sterile spreader.

Construction of Human Naïve VH_{μs} Library

Amplification of Human HCDR3 Repertoire

To generate the human HCDR3 library, the PCR fragments of the primary amplification of the heavy chain variable regions (see section Amplification of heavy chains) were amplified by using sense primers located in the framework 4 and antisense primers located in the framework 3 of VH of the heavy chain variable regions. The oligonucleotide primers for the amplification of HCDR3 are described in Table 2.

A. primary amplification

| | |
|---|---|
| ExBack1 | 5′-GACACGGCCGTNTATTACTGTG-3′ |
| ExBack2 | 5′-GACACGGCCGTNTATTATTGTG-3′ |
| ExBack3 | 5′-GACACGGCTGTRTATTTCTGTG-3′ |
| ExFor1 | 5′-GACCAGGGTBCCCTGGCCCCA-3′ |
| ExFor2 | 5′-GACCGTGGTYCCTTGGCCCCA-3′ |
| ExFor3 | 5′-GACCAGGGTGCCACGGCCCCA-3′ |

B. Secondary amplification (5′->3′)

| | |
|---|---|
| ExBack1-SfiI | GTCCTCGCAACTGCGGCCCAGCCGGCCATGGCCGACA |
| | CGGCCGTNTATTACTGTG |
| ExBack2-SfiI | GTCCTCGCAACTGCGGCCCAGCCGGCCATGGCCGACA |
| | CGGCCGTNTATTATTGTG |
| ExBack3-SfiI | GTCCTCGCAACTGCGGCCCAGCCGGCCATGGCCGACA |
| | CGGCTGTRTATTTCTGTG |
| ExFor1-NotI | GAGTCATTCTCGACTTGCGGCCGCTGAACCGCCTCCG |
| | ACCAGGGTBCCCTGGCCCCA |
| ExFor2-NotI | GAGTCATTCTCGACTTGCGGCCGCTGAACCGCCTCCG |
| | ACCGTGGTYCCTTGGCCCCA |
| ExFor3-NotI | GAGTCATTCTCGACTTGCGGCCGCTGAACCGCCTCC |
| | GACCAGGGTGCCACGCCCCA |

The PCR reaction was performed in 50 μl of reaction volume with 25 pmole of each primer and 1 ng or 0.1 ng of purified DNA. The reaction conditions for the PCR were 10 min at 94° C., followed by 30/30/60 sec at 94/55/72 ° C. for 25 cycles, and 10 min at 72° C. All reactions were performed with 2.5 mM MgCl₂, 200 μM dNTP (Roche Diagnostics, Brussels, Belgium) and 2.5 U AmpliTaq Gold DNA polymerase (Applied Biosystems, Lennik, Belgium).

All PCR products were separated on 4% low-melting agarose gel and the DNA was eluted using the QIAquick gel extraction kit or QIAEXII (Qiagen, Westburg, Leusden, The Netherlands).

A reamplification of the obtained HCDR3 fragments with SfiI and NotI restriction tagged oligonucleotides was performed to generate the VH_{μs} library (Table 2). Combinations of primers were used. The PCR reaction was performed in 50 μl of reaction volume with 25 pmole of each primer and 1 ng of purified DNA. The reaction conditions for the PCR were the same as described above. All PCR products were separated on 1.5% low melting agarose gel and the DNA was eluted using the QIAquick gel extraction kit or QIAEXII (Qiagen, Westburg, Leusden, The Netherlands).

Electroporation of Bacterial Cells.

The procedures used here are the same as those described for the VH library. 90-260 ng fragment was ligated into 450-1300 ng of the pAX001 display vector linearized with SfiI and NotI, using T4 DNA ligase (Promega, Leiden, The Netherlands).

Conclusion:

From the 10 donors, 10 individual libraries were constructed for both IgG and IgM repertoires. This was done for VH and VH_{μs} (i.e. the HDCR3 library in microscaffold format), resulting in 40 libraries. After assessing-their quality, these individual libraries finally were pooled together to obtain four libraries: VH/IgM, VH/IgG, VH_{μs}/IgM and VH_{μs}/IgG.

### Example 2

### Construction of Llama Dedicated VHH and VHH_{μs} Libraries

Llama Immunization

Two llamas were immunized according to animal welfare regulations. A cocktail of antigens (IL-6, TNFalpha, IgE, Von Willebrandt Factor, I domain, Ghrelin, Motilin, GpIb, Carcino Embryonic Antigen (CEA)) was formulated in Specol adjuvant. The immunization scheme used is depicted in Table 3.

TABLE 3
Immunization scheme of llamas

| | Immunization | | Sampling | |
|---|---|---|---|---|
| Day | Llama 2 | Llama 4 | Llama 2 | Llama 4 |
| 0 | Max dose | Max dose | 150 ml blood | 150 ml blood |
| 7 | Max dose | | | |
| 14 | ½ dose | | | |
| 21 | ½ dose | Max dose | | |
| 28 | ½ dose | | 10 ml blood | 10 ml blood |
| 35 | ½ dose | | | |
| 39 | | | 150 ml blood + | |
| | | | lymph node | |
| 42 | | ½ dose | 150 ml blood | |
| 45 | | | 150 ml blood | |
| 70 | | ½ dose | | |
| 74 | | | | 150 ml blood + |
| | | | | lymph node |
| 80 | | | | 150 ml blood |

VHH Library Construction

RNA was isolated from blood and lymph nodes according the method described by Chomzcynski and Sacchi, 1987. cDNA was prepared on 100 μg total RNA with M-MLV Reverse Transcriptase (Gibco BRL) and a hexanucleotide random primer (Amersham Biosciences) or oligo-dT primer as described before (de Haard et al., 1999). The cDNA was purified with a phenol/chloroform extraction combined with an ethanol precipitation and subsequently used as template to specifically amplify the VHH repertoire. The complete heavy chain derived IgG genes from the Cameloid heavy-chain antibodies (1.3 kB) and the conventional antibodies (1.65 kB) were amplified with oligo-dT primer combined with FR1-primer ABL013 (5′-GAGGTBCARCTGCAGGASTCYGG-3′) on oligo-dT primed cDNA as template according to the methods described in EP01205100.9. The heavy chain antibody derived IgG amplicon was gel purified and used for cloning after digestion with PstI (introduced in FR1-primer) and BstEII, which naturally occurs in the FR4-region.

Alternatively, the repertoire was amplified in a hinge-dependent approach using two IgG specific oligonucleotide primers. In a single PCR reaction FR1-primer ABL013 was combined with a short (5′-AACAGTTAAGCTTCCGCTTGCGGCCGCGGAGCTGGGGTCTTCGCTGTGGTGCG-3′) or long (5′-AACAGTTAAGCTTCCGCTTGCGGCCGCTGGTTGTGGTTTTGGTGTC TTGGGTT-3′) hinge primer known to be specific for the amplification of heavy-chain variable region gene segments.

A PstI (bold) and NotI (bold underlined) restriction site was introduced within the FR1 and hinge primers respectively, to allow cloning. Subsequently, the DNA fragments were ligated into the PstI-BstEII or PstI-NotI digested phagemid vector pAX001, which is identical to pHEN1 (Hoogenboom et al., 1991), but encodes a carboxyterminal (His)₆- and c-myc-tag for purification and detection, respectively. The ligation mixture was desalted on a Microcon filter (YM-50, Millipore) and electroporated into E. coli TG1 cells to obtain a library. The transformed cells were grown overnight at 37° C. on a single 20×20 cm plate with LB containing 100 μg/ml ampicillin and 2% glucose. The colonies were scraped from plates using 2×TY medium and stored at −80° C. in 20 % glycerol.

Finally, after electroporation of TG1 cells, 6 immune libraries were obtained. These are described in the Table 4.

TABLE 4
features of the 6 immune libraries

| | | | |
|---|---|---|---|
| Llama | Source | Size | Total |

| | | | | |
|---|---|---|---|---|
| Llama 2 | PBL time 1 | 1.87 | 10⁷ | Σ = 1.16 10⁹ |
| Llama 2 | PBL time 2 | 1.13 | 10⁹ | |
| Llama 2 | Lymph node | 1.26 | 10⁷ | |
| Llama 4 | PBL time 1 | 1.75 | 10⁸ | Σ = 2.26 10⁸ |
| Llama 4 | PBL time 2 | 2.17 | 10⁶ | |
| Llama 4 | Lymph node | 4.9 | 10⁷ | |

HCDR3 Amplification

DNA was prepared from all 6 immune libraries and used as template. The backward and forward primers were designed in order to maximally cover the HCDR3 repertoire.

FR3—Backward Primers

| | |
|---|---|
| GACACGGCCGBCTATTACTG | Exback1 |
| GACACGGCCGTTTATWACTG | Exback2 |
| GACACGGCCGTGTATTAYTG | Exback3 |
| GACACGGCCGTCTATTWTTG | Exback4 |
| GACACGGCCGWTTATTATTG | Exback5 |
| GACACGGCCATYTATTWCTG | Exback6 |
| GACACGGGACTYTATTACTG | Exback7 |
| aa:D T A V Y Y C | |

FR4—Forward Primers

| | | |
|---|---|---|
| A | GGGGCCAGGGVACYCAGGTC | |
| compl: | GACCTGRGTBCCCTGGCCCC | Exfor1 |
| B | GGGGCMAAGGGACCMAGGTC | |
| compl: | GACCTKGGTCCCTTKGCCCC | Exfor2 |
| C | GRGGSCCGGGGACCCAGGTC | |
| compl: | GACCTGGGTCCCCGGSCCYC | Exfor3 |
| D | GGGGDCAGGGGACCCAGGTC | |
| compl: | GACCTGGGTCCCCTGHCCCC | Exfor4 |
| E | ACGGCCAGGGGACCCAGGTC | |
| compl: | GACCTGGGTCCCCTGGCCGT | Exfor5 |
| aa: | G Q G T Q V | |

Combinations of primers were made to decrease the number of amplifications (Table 5).

TABLE 5
Combinations of primers used in the first
amplification of the HCDR3 regions

| | |
|---|---|
| P1 | Exback 3 |
| | Exback 4 |
| | Exback 5 |
| P2 | Exback 1 |
| | Exback 2 |
| | Exback 6 |
| | Exback 7 |
| P3 | Exfor 1 |
| | Exfor 2 |
| | Exfor 3 |
| | Exfor 4 |
| P4 | Exfor 5 |

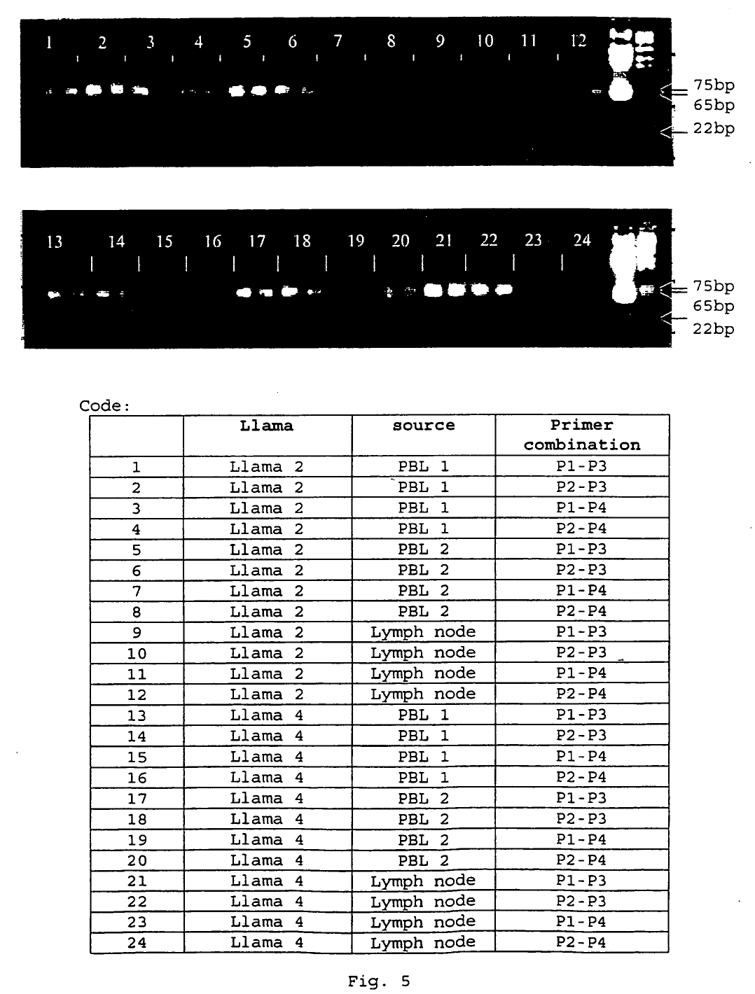

For each template 4 reactions were performed using different forward/backward primer combinations (P1-P3, P1-P4, P2-P3, P2-P4). Primary amplifications were carried out in 50 μl volume using 2.5 units Amplitaq Gold (Applied Biosystems), 0.2 mM dNTP, 25 pmol backward primer, 25 pmol forward primer, 1 or 0.1 ng template, following the program depicted in Table 6. Amplifications were analyzed on 4% low-melting agarose. The resulting products are shown in FIG. 5.

TABLE 6
program used in the first amplification.

| Time | | Temperature (° C.) | Cycling |
|---|---|---|---|
| 10 | min | 94 | |
| 30 | sec | 94 | 25 times |
| 30 | sec | 55 | |
| 1 | min | 72 | |
| 10 | min | 72 | |

The PCR products of the primary amplifications were gel purified and used as template for the secondary amplification reactions using the following primers.

FR3—Backward Primers

GTCCTCGCAACTGCGCCCCAGCCGGCCATGGCCGACACGGCCGBCTATTA
CTG
(Exback1sfi)
GTCCTCGCAACTGCGGCCCAGCCGGCCATGGCCGACACGGCCGTTTATWA
CTG
(Exback2sfi)
GTCCTCGCAACTGCGGCCCAGCCGGCCATGGCCGACACGGCCGTGTATTA
YTG
(Exback3sfi)
GTCCTCGCAACTGCGGCCCAGCCGGCCATGGCCGACACGGCCGTCTATTW
TTG
(Exback4sfi)
GTCCTCGCAACTGCGGCCCAGCCGGCCATGGCCGACACGGCCGWTTATTA
TTG
(Exback5sfi)
GTCCTCGCAACTGCGGCCCAGCCGGCCATGGCCGACACGGCCATYTATTW
CTG
(Exback6sfi)
GTCCTCGCAACTGCGGCCCAGCCGGCCATGGCCGACACGGGACTYTATTA
CTG
(Exback7sfi)

FR4—Forward Primers

GAGTCATTCTCGACTTGCGGCCGCTGAACCGCCTCCGACCTGRGTBCCCT
GGCCCC
(Exfor1not)
GAGTCATTCTCGACTTGCGGCCGCTGAACCGCCTCCGACCTKGGTCCCTT
KGCCCC
(Exfor2not)
GAGTCATTCTCGACTTGCGGCCGCTGAACCGCCTCCGACCTGGGTCCCCG
GSCCYC
(Exfor3not)
GAGTCATTCTCGACTTGCGGCCGCTGAACCGCCTCCGACCTGGGTCCCCT
GHCCCC
(Exfor4not)
GAGTCATTCTCGACTTGCGGCCGCTGAACCGCCTCCGACCTGGGTCCCCT
GGCCGT
(Exfor5not)

FR4—Forward Primers with Introduction CYS (104)

GAGTCATTCTCGACTTGCGGCCGCTGAACCGCCTCCGACCTGRGTBCCCT
GGCACCA
(Exfor1cysWnot)
GAGTCATTCTCGACTTGCGGCCGCTGAACCGCCTCCGACCTGRGTBCCCT
GGCACCT
(Exfor1cysRnot)
GAGTCATTCTCGACTTGCGGCCGCTGAACCGCCTCCGACCTKGGTCCCTT
KGCACCA
(Exfor2cysWnot)
GAGTCATTCTCGACTTGCGGCCGCTGAACCGCCTCCGACCTGGGTCCCCG
GSCACCA
(Exfor3cysWnot)
GAGTCATTCTCGACTTGCGGCCGCTGAACCGCCTCCGACCTGGGTCCCCG
GSCATCT
(Exfor3cysRnot)
GAGTCATTCTCGACTTGCGGCCGCTGAACCGCCTCCGACCTGGGTCCCCT
GHCACCA
(Exfor4cysWnot)
GAGTCATTCTCGACTTGCGGCCGCTGAACCGCCTCCGACCTGGGTCCCCT
GGCAGTA
(Exfor5cysWnot)

To decrease the number of these amplifications, combinations of primers were used as depicted in table 7:

TABLE 7
Combinations of primers used in the second
amplification of the HCDR3 regions

| | |
|---|---|
| P1 | Exback 3 Sfi |
| | Exback 4 Sfi |
| | Exback 5 Sfi |
| P2 | Exback 1 Sfi |
| | Exback 2 Sfi |
| | Exback 6 Sfi |
| | Exback 7 Sfi |
| P3 | Exfor 1 Not |
| | Exfor 2 Not |
| | Exfor 3 Not |
| | Exfor 4 Not |
| P4 | Exfor 5 Not |
| P3′ | Exfor1cysWnot |
| | Exfor1cysRnot |
| | Exfor2cysWnot |
| | Exfor3cysWnot |
| | Exfor3cysRnot |
| | Exfor4cysWnot |
| P4′ | Exfor5cysWnot |

For each template 8 reactions were performed using different forward/backward primer combinations (P1-P3, P1-P4, P2-P3, P2-P4, P1-P3′, P1-P4′, P2-P3′, P2-P4′). The secondary amplifications were carried out in 50 μl volume using 2.5 units Amplitaq Gold (Applied Biosystems), 0.2 mM dNTP, 25 pmol backward primer, 25 pmol forward primer, 1 ng template. The reactions were carried out in quadruplicate giving a final volume of 200 μl. The following program (table 8)was used:

TABLE 8
Program used in the second amplification

| | | Temperature | |
|---|---|---|---|
| Time | | (° C.) | Cycling |
| 10 | min | 94 | |
| 30 | sec | 94 | 25 times |
| 30 | sec | 55 | |
| 1 | min | 72 | |
| 10 | min | 72 | |

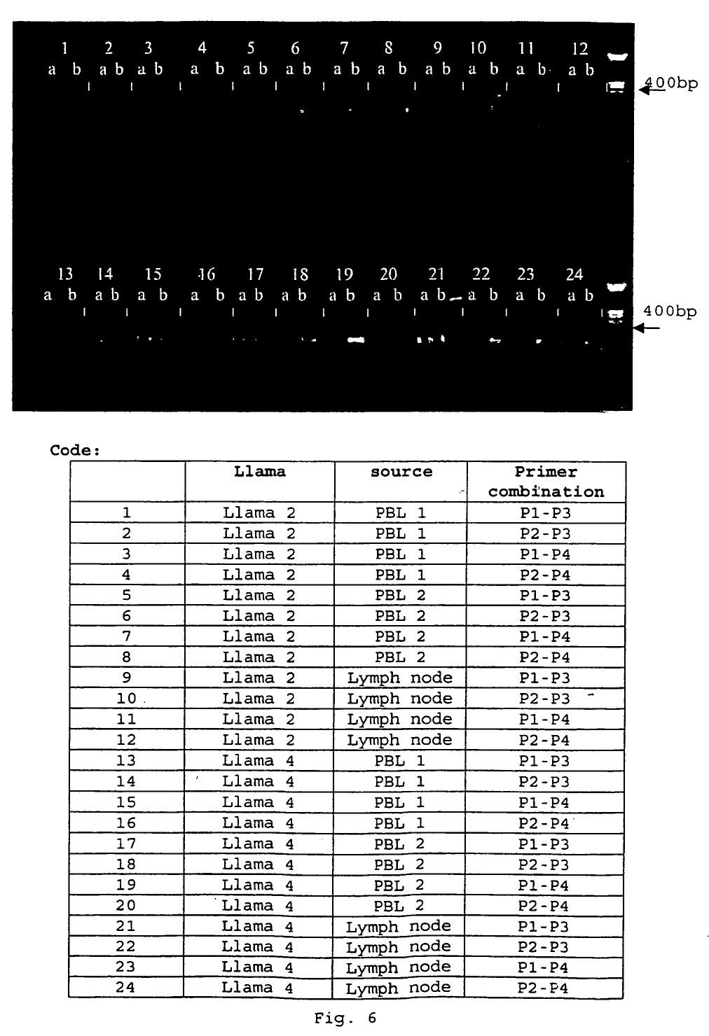

All PCR products were separated on 1.5% low-melting agarose gel (FIG. 6) and digested with Not1 and Sfi1. Fragments derived from the same template source (llama, tissue) were pooled. Product with or without Cysteine in FR4 were kept separate. 90-260 ng fragment was ligated to 450-1300 ng of the pAX001 display vector, linearized with Sfi1 and Not1. The diversity obtained after electroporation of TG1 cells is described in the Table 9.

TABLE 9
VHH_{μs} libraries
pAX001

| Llama | Source | Size | | Total |
|---|---|---|---|---|
| Llama 2 | PBL time 1 | 9 | 10⁵ | Σ = 7 10⁷ |
| Llama 2 | PBL time 2 | 7.2 | 10⁶ | |
| Llama 2 | Lymph node | 6.12 | 10⁷ | |
| Llama 2 + Cysteine | PBL time 1 | 1.26 | 10⁷ | Σ = 3.66 10⁷ |
| Llama 2 + Cysteine | PBL time 2 | 1.2 | 10⁷ | |
| Llama 2 + Cysteine | Lymph node | 1.2 | 10⁷ | |

The percentage of insert containing clones was determined in PCR using the M13 reverse and gene3 forward primer. The results are presented in Table 10.

TABLE 10
Percentage insert of VHH HCDR3 libraries

| | % insert |
|---|---|
| PBL time 1 | 84 |
| PBL time 2 | 81 |
| Lymph node | 100 |
| PBL time 1 + Cysteine | 97 |
| PBL time 2 + Cysteine | 97 |
| Lymph node + Cysteine | 94 |

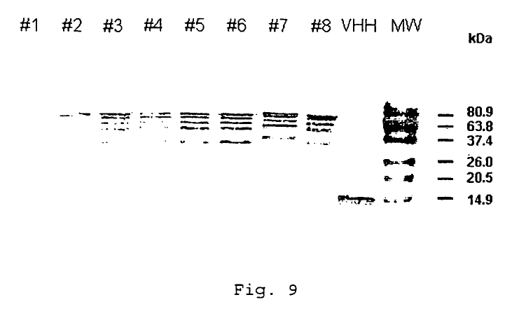

A number of clones of the library was picked randomly and used for expression of the HCDR3-gene 3 fusion. Each clone was grown in 1 ml of culture (2TY/ampicillin/0.1% glucose) at 37° C. and induced at an OD600 of 0.9 by addition of IPTG to a final concentration of 1 mM. After 4 hours continued growth the cells were harvested by centrifugation and dissolved in 200 ml Laemmli buffer; 5 ml was loaded on 15% PAGE after boiling for 5 minute. After electroblotting the HCDR3-derived products were detected with the anti-MYC antibody 9E10, which recognizes the carboxyterminal peptide tag (see FIG. 9).

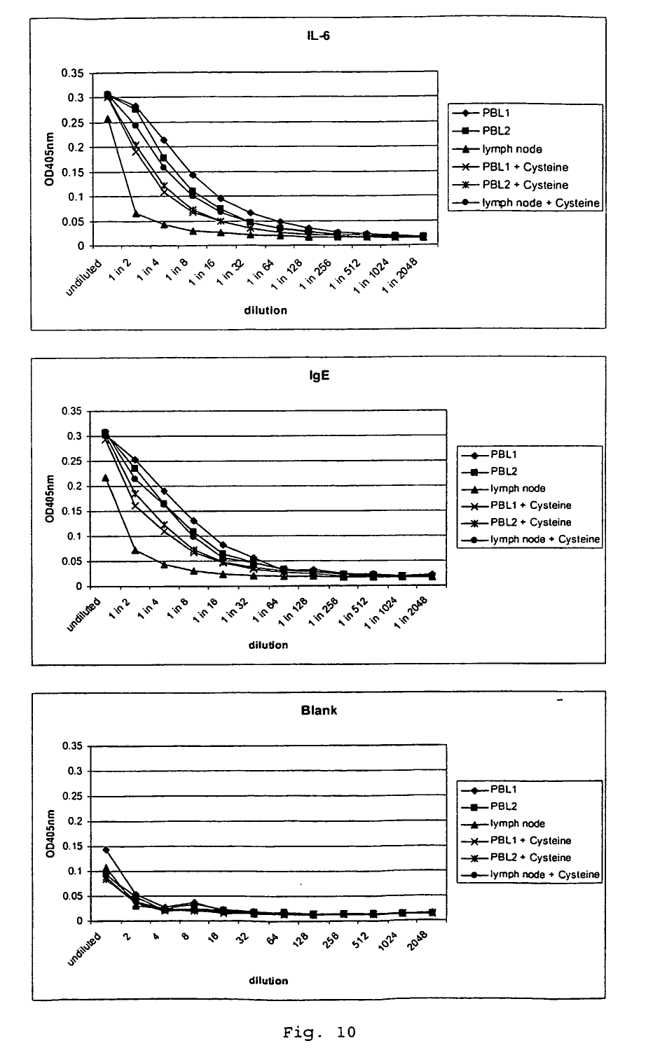

The quality of the pAX1-library was analyzed by a phage ELISA, in which polyclonal phage prepared from the non-selected library were tested in dilution series on the antigens IL-6, TNFalpha, IgE and CEA. Bound phage was detected with an anti-phage M13 gene8 mAB (Amersham Biosciences) Specific signals were found with all tested antigens, while no response was seen against the irrelevant antigen β-casein (see FIG. 10).

### Example 3

### Selection on Chemokine Receptors CXCR4 and CCR5 by using Naïve VH, VH_{μs} and VHH_{μs} Libraries

Human glioma cells expressing CD4 and human chemokine receptors CXCR4 or CCR5 (Centralized Facility for AIDS Reagents, NIBSC, UK) were grown in 85% DMEM, 15% heat inactivated foetal calf serum, 300 μg/ml G418 and 1 μg/ml of puromycine to confluent monolayers in 6 well culture plates. 10¹³ phages/phagemid particles of the VH, VHH_{μσ}and VH_{μs} library with a diversity of 10¹⁰ unique clones for human libraries and 10⁷ unique clones for the llama library were incubated in 1 ml culture medium with the adherent glioma cells for 3.5 hrs at 4° C. Following 5 washes with culture medium with 5 minute incubation between the washes, bound phages/phagemids were eluted for 10 minutes with 0.1N glycine pH 2.2. After neutralization with 1M Tris-HCl buffer pH 8.1, eluted phages/phagemids were used to infect exponentially growing E. coli TG1 cells. Bacteria were plated on LB agar plates with 100 μg/ml ampicillin or tetracyclin and 2% glucose. Phages were prepared from bacteria and phagemids were rescued by using M13K07 helper phages to use in a next selection round. Different biopanning strategies were performed with CXCR4 or CCR5 expressing human glioma cells, the corresponding human glioma cells that were not expressing CXCR4 and CCR5 and other cell types expressing CXCR4 and CCR5 to identify sequences that were specifically binding to CXCR4 and CCR5.

After the biopanning procedures individual phages/phagemids were tested for their reactivity to CXCR4 and CCR5 expressing cells in an ELISA assay. Cells were grown to monolayers in 96 well plates overnight. After gentle washing with PBS, the plates were blocked with 2% BSA in PBS for 2 hrs. Phages/phagemids were added to the plates and allowed to bind to the cells for 2 hrs at 4° C. Unbound phages and phagemids were removed by gentle washing with PBS. The binding of the phages/phagemids was detected with HRP conjugated ant-M13 antibody and orthophenylenediamine-H₂O₂ as substrate. Plates were analyzed in a microtiterplate reader at 492 nm. Phages/phagemids binding specifically to the CXCR4 and CCR5 expressing cells were obtained by using different biopanning strategies on different cells.

### Example 4

### Biopanning Using a Dedicated VHH Library

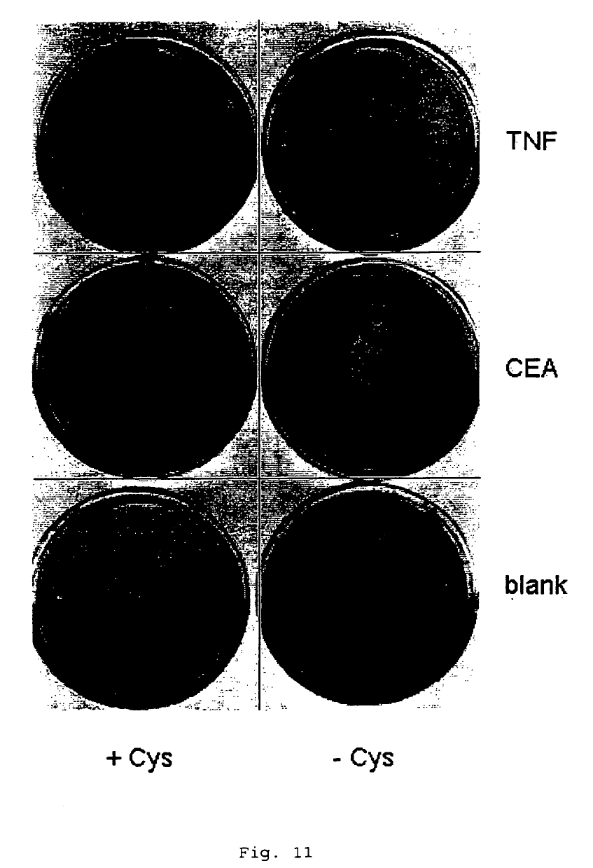

Libraries were grown and infected with helperphage M13K07 to obtain phages expressing HCDR3 on the tip of the phage. Phages were purified and used in biopanning experiments. 100∝1 of antigen at a concentration of 5 μg/ml (in PBS) was coated in microtiterplates during 16 hours at 4° C. Plates were blocked for 2 hours at room temperature using 1% skimmed milk. 50 μl purified phages were mixed with 50 μl. 0.2% skimmed milk and incubated with the antigen for 2 hours at room temperature. Non-bound phages were washed away using PBS+0.05% Tween-20. Specific phages were eluted using 50 μl 0.1 M glycine pH 2.5 and neutralized with 50 μl 1M Tris-HCl pH 7.5. Antigen specific phage were eluted as could be concluded from the numbers of clones obtained from antigen coated wells compared with those from β-casein coated wells leading to enrichment factors of more than 100 for both the disulfide bridge containing micro-scaffold library and the one lacking this bridge (see FIG. 11). The results are shown in Table 11.

TABLE 11
Selection of HCDR3 fragments

| | | | | Llama 2 PBL1, |
|---|---|---|---|---|
| Selection 1° | | | Llama 2 PBL1, | PBL2, lymph |
| round | Concentration | | PBL2, lymph | node |
| Target | (μg/ml) | | node | + cysteine |
| TNF-alpha | 10 | μg/ml | 10⁴ | 2 10⁴ |
| | 0 | μg/ml | 3 10³ | 4 10³ |
| CEA | 10 | μg/ml | 1.5 10⁴ | 2 10⁴ |
| | 0 | μg/ml | 3 10³ | 4 10³ |
| IgE | 10 | μg/ml | 5 10⁴ | 6 10⁴ |
| | 0 | μg/ml | 3 10³ | 4 10³ |
| IL-6 | 10 | μg/ml | 9 10³ | 10⁴ |
| | 0 | μg/ml | 3 10³ | 4 10³ |

### Example 5

### Characterization of the HCDR3 Length Distribution from Dedicated VHH Library

To assess the length distribution of the HCDR3 in the library derived from the immunized llama a PCR was performed by PCR amplification (using the protocol of Table 6 and with 1 ng of plasmid template) with the FAM labeled gene 3 primer combined with the different pools of FR3-based backward primers (Table 5). 1 μl of the PCR was added to 19 μl deionized water. 1 μl of the diluted PCR products was mixed with 10 μl formamide-size standard-mix, containing 1 ml of Hi-Di™ Formamide and 17 μl of GeneScan™-400HD ROX or 500 ROX (Applied Biosystems, Foster City, Calif. 94404, USA) . The samples were heated for 5 minutes at 95° C. and placed on ice for at least 5 minutes before loading on the ABI 3700 sequencing machine (Applied Biosystems).

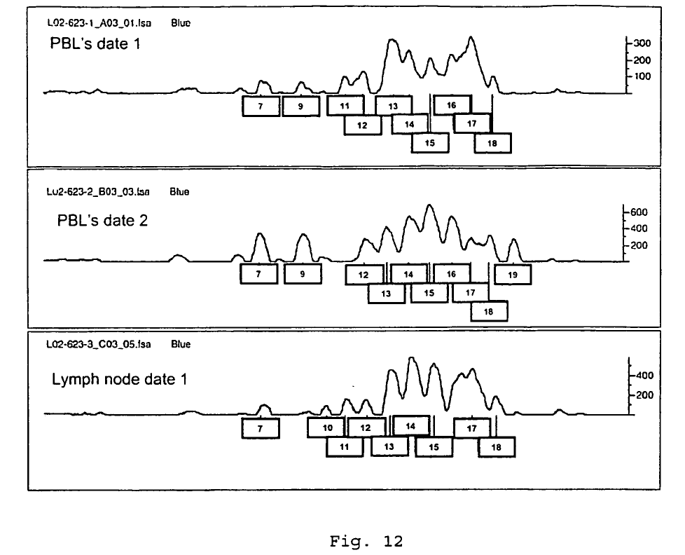

The obtained chromatograms showed that the length of HCDR3 varied in triplets, i.e. codons, and that the major peaks were obtained between 13 and 17 amino acid residues (see FIG. 12).

Methods

Vector Construction.

Different display vectors were designed and digested with Sfi1 and Not1 restriction enzymes to have in frame cloning of the HCDR3 PCR products:

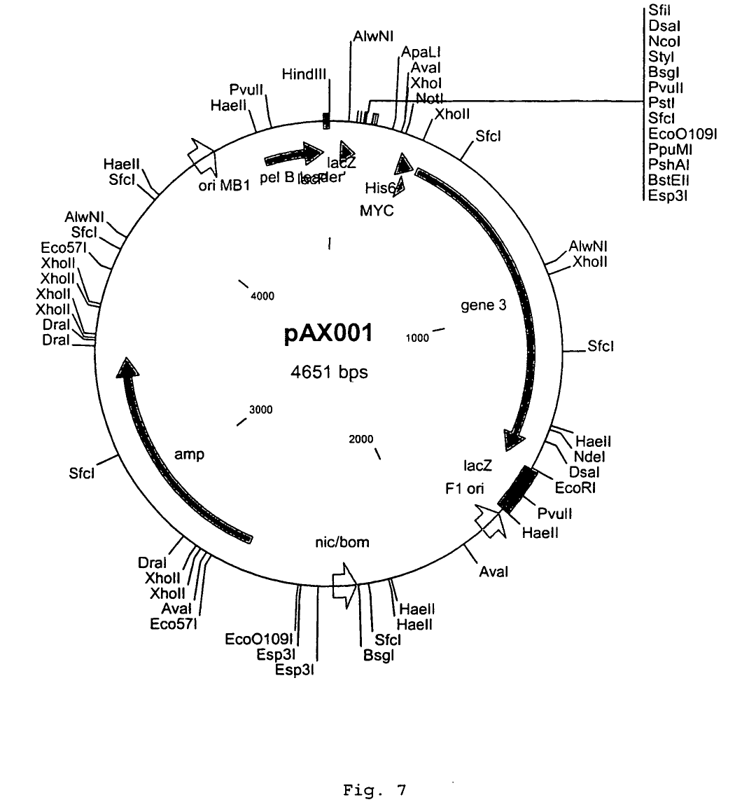

1. pAX001 (FIG. 7): identical to pHEN1 (Hoogenboom et al., 1991), but adapted for: enables expression of a HCDR3 in fusion with a HIS-tag, a c-myc-tag and pIII. An amber stopcodon between the c-myc tag and the pIII sequence allows expression of the full fusion product when expressed in a suppressor strain. When expressed in a non-suppressor strain soluble HCDR3 in fusion with a HIS-tag and a c-myc-tag can be obtained.

2. pAX007: enables expression of a HCDR3 in fusion with a c-myc-tag, a HIS-tag and pIII. The pAX001 display vector was modified so that the amber stopcodon was replaced by a codon encoding Glu.

The following mutagenesis primer was designed: 5′ACTCTCGAGATCAAACGGGCGGCCGCAGAACAAAAACTCATCTCAGAAGAGGAT CTGAATGGGGCCGCACATCATCATCACCATCACGGGGCCGCAGAAACTGTTGAAAG TTGTTTAGCA3′ and used to modify the pAX001 display vector.

3. pAX008: enables expression of a HCDR3 in fusion with a c-myc-tag, a HIS-tag and the c-terminal domain of pIII, anchoring the fusion product in the phage coat.

The following mutagenesis primer was designed: 5′ACTCTCGAGATCAAACGGGCGGCCGCAGAACAAAAACTCATCTCAGAAGAGGAT CTGAATGGGGCCGCACATCATCATCACCATCACGGGGCCGCAGGTGGTGGCTCTGG TTCCGGTGA3′ and used to modify the pAX001 display vector.

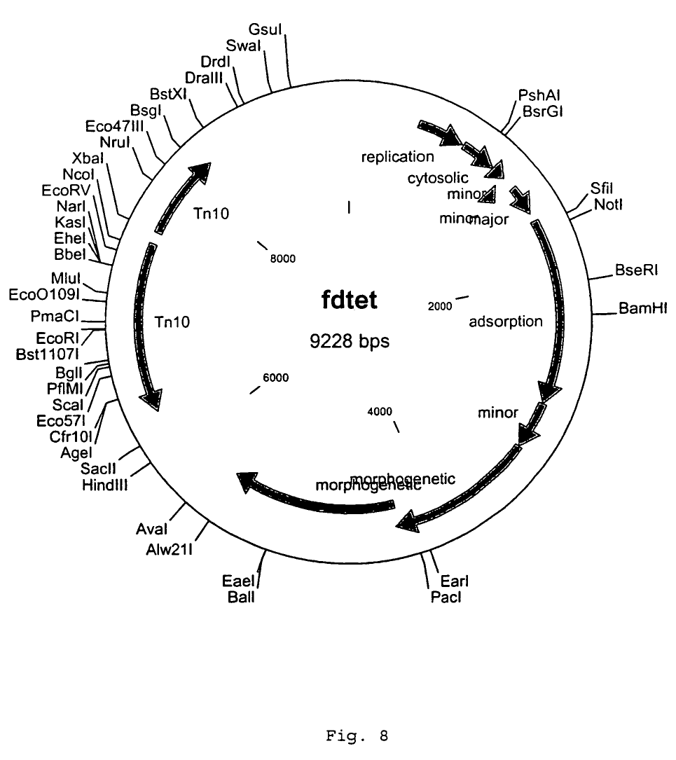

4. phage fd-tet (FIG. 8): enables multivalent expression of a HCDR3 in fusion with pIII (Zacher et al., 1980)

Generation of Electrocompetent Cells

TG-1 cells were cultured in 1 L 2TY medium containing 16 g/L Tryptone (Difco, Becton Dickinson, San Diego, USA), 10 g/L yeast extract (Difco, Becton Dickinson, San Diego, USA) and 5 g/L NaCl (Merck Eurolab, Overijse, Belgium) at 37° C. at 200-250 rpm until an OD_{600 nm} of 0.6-0.9 was reached. Cultures were placed on ice for 30-60 min and then centrifuged for 10 min at 4° C. at 4000 rpm in a GS-3 rotor. Cells were suspended in an equal volume of ice-cold distilled water, incubated on ice for 30-60 min and centrifuged. Cells were then suspended in half a volume of ice-cold distilled water, incubated on ice for 30-60 min and centrifuged. Next, cells were suspended in 10% glycerol, incubated on ice for 30-60 min and centrifuged. In the last step, cells were suspended in 1 ml 10% glycerol and stored on ice until further use.

### REFERENCES

Adib-Conquy M., Gilbert M., and Avrameas S. (1998). Int. Immunol. 10, 341-346.

Arbabi Ghahroudi M., Desmyter A., Wyns L., Hamers R., and Muyldermans S. (1997). FEBS Lett. 414, 521-526.

Cai X. and Garen A. (1996). Proc. Natl. Acad. Sci. USA 93, 6280-6285.

Chen C., Stenzel-Poore M. P., and Rittenberg M. B. (1991). J. Immunol. 147, 2359-2367.

Chomzcynski P. and Sacchi N. (1987), Anal. Biochem. 162, 156-159.

Davies J., and Riechmann L. (1994) FEBS Lett. 339, 285-290.

Decker D. J., Boyle N. E., Koziol J. A., and Klinman N. R. (1991) J. Immunol. 146, 350-361.

De Haard H. J., van Neer N., Reurs A., Hufton S. E., Roovers

R. C., Henderickx P., de Bruine A. P., Arends J. W., and Hoogenboom H. R. (1999), J. Biol. Chem. 274, 18218-18230.

Deng Y. J., and Notkins A. L. (2000) Clin. Exp. Immunol. 119, 69-76.

Desmyter A., Transue T. R., Ghahroudi M. A., Thi M. H., Poortnmans F., Hamers R., Muyldermans S., and Wyns L. (1996). Nat. Struct. Biol. 3, 803-811.

Desmyter A., Decanniere K., Muyldermans S., and Wyns L. (2001) J. Biol. Chem. 276, 26285-26290.

Ditzel H. J., Itoh K., and Burton D. R. (1996). J. Immunol. 157, 739-749.

Hamers-Casterman C., Atarhouch T., Muyldermans S., Robinson G., Hamers C., Songa E. B., Bendahman N., and Hamers R. (1993) Nature 363, 446-448.

Hoogenboom H. R., Griffith A. D., Johnson K. S., Chiswell D. J., Hudson P., and Winter G. (1991), Nucleic Acids Res. 19, 4133-4137.

Hufton S. E., van Neer N., van den Beuken T., Desmet J., Sablon E., and Hoogenboom H. R. (2000). FEBS Lett. 475, 225-231.

Kabat E. A., and Wu T. T. (1991). J. Immunol. 147, 1709-1719.

Larocca D., Kassner P. D., Witte A., Ladner R. C., Pierce G. F., and Baird A. (1999). FASEB J. 13, 727-734.

Ma X-L, Tao P. S. and Lefer A. M., (1991), f. 88, 1237-1343.

Padlan E. A. (1994). Mol. Immunol. 31, 169-217.

Painter R. G., Sage H. J., and Tanford C. (1972). Biochemistry 11, 1327-1337.

Marks J. D., Tristem M., Karpas A., and Winter G. (1991) Eur. J. Immunol. 21, 985-991.

Mian I. S., Bradwell A. R., and Olson A J. (1991) J. Mol. Biol. 217, 133-151.

Nuttall S. D., Krishnan U. V., Hattarki M., De Gori R., Irving R. A., and Hudson P. J. (2001) Mol. Immunol. 38, 313-326.

Nuttall S. D., Krishnan U. V., Doughty L., Nathanielsz A., Ally N., Pike R. N., Hudson P. J., Kortt A. A., and Irving R. A. (2002). FEBS Lett. 516, 80-86.

Raaphorst F. M., Raman C. S., Tami J., Fischbach M., and Sanz I. (1997). Int. Immunol. 9, 1503-1515.

Reiter Y;, Schuck P., Boyd L. F., and Plaksin D. (1999). J. Mol. Biol. 290, 685-698.

Roux K. H., Greenberg A. S., Greene L., Strelets L., Avila D., McKinney E. C., and Flajnik M. F. (1998) Proc. Natl. Acad. Sci. USA 95, 11804-11809.

Saphire E. O., Parren P. W., Pantophlet R., Zwick M. B., Morris G. M., Rudd P. M., Dwek R. A., Stanfield R. L., Burton D. R., and Wilson I. A. (2001) Science 293, 1155-1159.

Shapiro S. D., Kobayashi D. K., and Ley T. J. (1993). J. Biol. Chem. 268, 23824-23829.

Smith J. W., Tachias K., and Madison E. L. (1995) J. Biol. Chem. 270, 30486-30490.

Taub R., Gould R. J., Garsky V. M., Ciccarone T. M., Hoxie J., Friedman P. A., and Shattil S. J. (1989) J. Biol. Chem. 264, 259-265.

Vaughan C. K., and Sollazzo M. (2001) Comb. Chem. High Throughput Screen. 4, 417-430.

van den Beucken T., van Neer N., Sablon E., Desmet J., Celis L., Hoogenboom H. R., and Hufton S. E. (2001). J. Mol. Biol. 310, 591-601

Ward E. S., Gussow D., Griffiths A. D., Jones P. T., and Winter G. (1989) Escherichia coli. Nature 341, 544-546.

Zacher A. N. 3^{rd}, Stock C. A., Golden J. W. 2^{nd}, and Smith G. P. (1980). Gene 9, 127-140.

1. Isolated polypeptide micro-scaffold consisting of a single domain antigen binding fragment, said micro-scaffold comprising a CDR1, a CDR2 or a CDR3 polypeptide sequence that is interconnected with fragments of the adjacent framework polypeptide sequences, which are arranged to form two anti-parallel β-strands, said scaffold anchoring and displaying a CDR1, a CDR2 or a CDR3 loop.

2. Isolated polypeptide micro-scaffold consisting of a single domain antigen binding fragment, said micro-scaffold comprising a CDR2 or a CDR3 polypeptide sequence that is interconnected with fragments of the adjacent framework polypeptide sequences, which are arranged to form two anti-parallel β-strands, said scaffold anchoring and displaying a CDR2 or a CDR3 loop.

3. Micro-scaffold as in claim 1, wherein said framework polypeptide sequences are selected from the group consisting of naturally occurring immunoglobulin framework polypeptide sequences, mutated naturally occurring framework polypeptide sequences, and artificial consensus framework polypeptide sequences.

4. Micro-scaffold as in claim 3, wherein said framework polypeptide sequences are mutated naturally occurring framework polypeptide sequences comprising cysteine residues at Kabat numbering positions 92 and 104 arranged to form a disulphide bridge crosslink for increasing the conformational stability of the anti-parallel β-strands.

5. Micro-scaffold as in claim 1, linked to a polypeptide suitable for presenting or expression of said micro-scaffold.

6. Micro-scaffold as in claim 5 wherein said polypeptide suitable for presenting or expression is a surface protein of a viral system with a solvent accessible N-terminus or C-terminus.

7. Micro-scaffold as in claim 1, wherein said CDR3 polypeptide sequence is a HCDR3 polypeptide sequence.

8. Isolated nucleotide sequence encoding the polypeptide micro-scaffold of claim 1.

9. Vector comprising the isolated nucleotide sequence of claim 8.

10. A CDR polypeptide library of micro-scaffolds according to claim 1, wherein the CDR1, the CDR2 or the CDR3 polypeptide sequences of a sufficient number of micro-scaffolds represent at least a significant fraction of a natural repertoire.

11. The CDR polypeptide library as in claim 10 wherein said sufficient number of micro-scaffolds lies between 10 and 10¹⁵.

12. A CDR nucleic acid library of micro-scaffold nucleotide sequences according to claim 8, wherein the CDR1, the CDR2 or the CDR3 nucleotide sequences of a sufficient number of micro-scaffolds represent at least a fraction of a natural repertoire.

13. A method for creating a micro-scaffold as in claim 1, comprising the steps of:
Providing a CDR1, a CDR2 or a CDR3 nucleotide sequence that is interconnected with fragments of its adjacent framework nucleotide sequences to obtain a micro-scaffold nucleotide sequence encoding the polypeptide micro-scaffold of claim 1, and
Express said micro-scaffold nucleotide sequence in a suitable system.

14. A method for creating a CDR library displaying loops of immunoglobulin domains, comprising the steps of:
Prepare a CDR nucleic acid library as in claim 12, and
Express said CDR nucleic acid library in a suitable system.

15. The method as in claim 14 wherein said suitable system is a viral system having a surface protein with a solvent accessible N-terminus or C-terminus.

16. A method to search, select or screen for immunoglobulin CDR1, CDR2 or CDR3 polypeptide sequences that bind to a given antigen or mixture of antigens, comprising the steps of:
Creating a CDR library with the method of claim 14 from the genetic information of an individual or group of individuals,
Select a CDR which binds to said antigen or mixture of antigens.

17. A method to search, select or screen for immunoglobulin CDR1, CDR2 or CDR3 polypeptide sequences that bind to a given antigen or mixture of antigens, comprising the steps of:
Creating a CDR library with the method of claim 14 from the genetic information of an individual or group of individuals,
Creating a VH, Fab, scFv or IgG library from the genetic information of said individual or said group of individuals, and
Selecting a CDR which binds to said antigen or mixture of antigens, in both said CDR library and said VH, Fab, scFv or IgG library.

18. A method to search, select or screen for immunoglobulin CDR1, CDR2 or CDR3 polypeptide sequences that bind to a given antigen or mixture of antigens, comprising the steps of:
Creating a CDR library with the method of claim 14 from the genetic information of an individual or group of individuals,
Creating a non-immunoglobulin grafted CDR library using a non-immunoglobulin scaffold that is arranged to comprise grafted CDR loops of said CDR library, and
Selecting a CDR which binds to said antigen or mixture of antigens, in both said CDR library and said non-immunoglobulin grafted CDR library.

19. The method of claim 16, wherein the individual or group of individuals are either immunised or naïve to the given antigen or mixture of antigens.

20. A method for identifying peptide molecules that are homologous to the sequence of the CDR sequences identified by the method of claim 16, comprising the steps of:
Providing micro-scaffolds consisting of a single domain antigen binding fragment, said micro-scaffold comprising a CDR1, a CDR2 or a CDR3 polypeptide sequence that is interconnected with fragments of the adjacent framework polypeptide sequences, which are arranged to form two anti-parallel β-strands, said scaffold anchoring and displaying a CDR1, a CDR2 or a CDR3 loop grafted with said CDR sequence and with its homologue, and
Testing whether said CDR homologue binds the antigen or mixture of antigens used to the same extent as the CDR sequences identified by the method of claim 16.

21. Micro-scaffold as in claim 2, wherein said framework polypeptide sequences are selected from the group consisting of naturally occurring immunoglobulin framework polypeptide sequences, mutated naturally occurring framework polypeptide sequences, and artificial consensus framework polypeptide sequences.

22. Micro-scaffold as in claim 2, linked to a polypeptide suitable for presenting or expression of said micro-scaffold.

23. Micro-scaffold as in claim 2, wherein said CDR3 polypeptide sequence is a HCDR3 polypeptide sequence.

24. Isolated nucleotide sequence encoding the polypeptide micro-scaffold of claim 2.

25. A CDR polypeptide library of micro-scaffolds according to claim 2, wherein the CDR1, the CDR2 or the CDR3 polypeptide sequences of a sufficient number of micro-scaffolds represent at least a significant fraction of a natural repertoire.

26. A method for creating a micro-scaffold as in claim 2, comprising the steps of:
Providing a CDR1, a CDR2 or a CDR3 nucleotide sequence that is interconnected with fragments of its adjacent framework nucleotide sequences to obtain a micro-scaffold nucleotide sequence encoding the polypeptide micro-scaffold of claim 2, and
Express said micro-scaffold nucleotide sequence in a suitable system.

27. A method for identifying peptide molecules that are homologous to the sequence of the CDR sequences identified by the method of claim 16, comprising the steps of:
Providing micro-scaffolds consisting of a single domain antigen binding fragment, said micro-scaffold comprising a CDR2 or a CDR3 polypeptide sequence that is interconnected with fragments of the adjacent framework polypeptide sequences, which are arranged to form two anti-parallel β-strands, said scaffold anchoring and displaying a CDR2 or a CDR3 loop grafted with said CDR sequence and with its homologue, and
Testing whether said CDR homologue binds the antigen or mixture of antigens used to the same extent as the CDR sequences identified by the method of claim 16.
